(19) United States
(12) Patent Application Publication
(10) Pub. No.: US 2023/0024917 A1
(43) Pub. Date: Jan. 26, 2023

(54) DEVICES AND METHODS FOR DEEP TISSUE TEMPERATURE MEASUREMENT USING OPTICAL SENSING

(71) Applicant: Cardiac Pacemakers, Inc., St. Paul, MN (US)

(72) Inventors: Jeffrey E. Stahmann, Ramsey, MN (US); Keith R. Maile, New Brighton, MN (US)

(21) Appl. No.: 17/864,899

(22) Filed: Jul. 14, 2022

Related U.S. Application Data
(60) Provisional application No. 63/223,197, filed on Jul. 19, 2021.

(51) Int. Cl. A61B 5/01; A61B 5/00

(52) U.S. Cl. CPC A61B 5/01; A61B 5/0075; A61B 5/7264; A61B 5/6846

(57) ABSTRACT

Embodiments herein relate to devices and methods for assessing deep tissue temperature using optical sensing. In an embodiment an optical temperature monitoring device is included having an optical emitter, wherein the optical emitter is configured to emit light at a first wavelength from 100 nm to 2000 nm. The optical temperature monitoring device also includes an optical detector configured to detect incident light. The optical temperature monitoring device can be configured so that the light from the optical emitter propagates at a depth of at least 1 cm through tissue as measured from a surface of the optical temperature monitoring device and back to the optical detector and the incident light detected by the optical detector is used to determine a temperature of the tissue at depths of at least 1 cm as measured from a surface of the optical temperature monitoring device. Other embodiments are also included herein.

This application claims the benefit of U.S. Provisional Application No. 63/223,197, filed Jul. 19, 2021, the content of which is herein incorporated by reference in its entirety.

### FIELD

Embodiments herein relate to devices and methods for assessing deep tissue temperature using optical sensing.

### BACKGROUND

Body temperature is considered to be one of the four key vital signs. Normal body temperature can range from 97.8° F. (36.5° C.) to 99° F. (37.2° C.) for a healthy adult varying depending on gender, recent activity, food and fluid consumption, time of day, and, in women, the stage of the menstrual cycle. Abnormal variations in body temperature can result from various pathologies such as fever, hypothermia, or hyperthermia.

Peripheral monitoring of temperature can provide insight into body temperature at shallow tissue depths of less than 1 cm. However, peripheral temperature monitoring can be limited by a variety of factors, including low extremity perfusion, iron deficiency, low cardiac output, vasoconstriction, hypothermia, variation in skin pigmentation, sickle cell anemia, fingernail polish or tattoo ink, and motion. In addition, peripheral temperature monitoring is particularly inaccurate at low ambient temperatures, where it tends to underestimate body temperature.

### SUMMARY

Embodiments herein relate to devices and methods for assessing deep tissue temperature using optical sensing. In a first aspect, an optical temperature monitoring device is included having an optical emitter, wherein the optical emitter is configured to emit light at a first wavelength from 100 nm to 2000 nm. The optical temperature monitoring device also includes an optical detector configured to detect incident light. The optical temperature monitoring device can be configured so that the light from the optical emitter propagates at a depth of at least 1 cm through tissue as measured from a surface of the optical temperature monitoring device and back to the optical detector and the incident light detected by the optical detector is used to determine a temperature of the tissue at depths of at least 1 cm as measured from a surface of the optical temperature monitoring device.

In a second aspect, in addition to one or more of the preceding or following aspects, or in the alternative to some aspects, the first wavelength can be from 590 nm to 650 nm.

In a third aspect, in addition to one or more of the preceding or following aspects, or in the alternative to some aspects, the optical temperature monitoring device can be configured to be implanted in a patient.

In a fourth aspect, in addition to one or more of the preceding or following aspects, or in the alternative to some aspects, the optical temperature monitoring device can be configured to be implanted in a subcutaneous tissue of a patient.

In a fifth aspect, in addition to one or more of the preceding or following aspects, or in the alternative to some aspects, the optical temperature monitoring device can be configured to be worn externally by a patient.

In a sixth aspect, in addition to one or more of the preceding or following aspects, or in the alternative to some aspects, wherein at least one of absorption, scattering and phase of the incident light detected by the optical detector can be used to determine the temperature of the tissue.

In a seventh aspect, in addition to one or more of the preceding or following aspects, or in the alternative to some aspects, the absorption is used to determine an optical density of the tissue and the optical density is used to determine the temperature of the tissue.

In an eighth aspect, in addition to one or more of the preceding or following aspects, or in the alternative to some aspects, wherein both an AC component and an DC component of the light detected by the optical detector are used to determine the temperature of the tissue.

In a ninth aspect, in addition to one or more of the preceding or following aspects, or in the alternative to some aspects, further can include a second optical emitter, wherein the second optical emitter is configured to emit the light at a second wavelength.

In a tenth aspect, in addition to one or more of the preceding or following aspects, or in the alternative to some aspects, the second wavelength is a near-infrared wavelength.

In an eleventh aspect, in addition to one or more of the preceding or following aspects, or in the alternative to some aspects, the second wave length is from 750 nm to 1500 nm.

In a twelfth aspect, in addition to one or more of the preceding or following aspects, or in the alternative to some aspects, the second wavelength has an optical density temperature coefficient that is less than the first wavelength.

In a thirteenth aspect, in addition to one or more of the preceding or following aspects, or in the alternative to some aspects, the temperature of the tissue is determined from a ratio of a parameter of the incident light on the detector originating from the light emitted at the first wavelength and a parameter of the incident light on the detector originating from the light emitted at the second wavelength.

In a fourteenth aspect, in addition to one or more of the preceding or following aspects, or in the alternative to some aspects, further can include: a housing, and a flexible member, wherein at least one of the optical emitter and the optical detector are disposed on the flexible member extending from the housing, and wherein the flexible member extends outward from the housing.

In a fifteenth aspect, in addition to one or more of the preceding or following aspects, or in the alternative to some aspects, the optical emitter and the optical detector are disposed on a substantially planar surface.

In a sixteenth aspect, in addition to one or more of the preceding or following aspects, or in the alternative to some aspects, the optical emitter is disposed from 1 cm to 10 cm away from the optical detector.

In a seventeenth aspect, in addition to one or more of the preceding or following aspects, or in the alternative to some aspects, further can include at least one of a pulse oximetry sensor, chemical sensor, posture sensor, and heart rate sensor.

In an eighteenth aspect, in addition to one or more of the preceding or following aspects, or in the alternative to some aspects, the temperature of the tissue is interpreted in view of at least one of heart rate, respiration, circadian rhythm, and posture.

In a nineteenth aspect, in addition to one or more of the preceding or following aspects, or in the alternative to some aspects, further can include a therapy device, wherein the therapy device is configured to deliver a therapy to a patient.

In a twentieth aspect, in addition to one or more of the preceding or following aspects, or in the alternative to some aspects, the optical emitter can include a first optical emitter element and a second optical emitter element, and wherein the first optical emitter element is spaced a distance away from the second optical emitter element.

In a twenty-first aspect, in addition to one or more of the preceding or following aspects, or in the alternative to some aspects, the temperature measured is a core temperature.

In a twenty-second aspect, in addition to one or more of the preceding or following aspects, or in the alternative to some aspects, the temperature of the tissue is calibrated with a second temperature measurement.

In a twenty-third aspect, a method of measuring a temperature of the tissue in a patient is included, the method including bringing an optical temperature monitoring device in contact with the tissue of the patient, the optical temperature monitoring device can include an optical emitter and an optical detector, and emitting light with the optical emitter into the tissue of the patient at a first wavelength, wherein the first wavelength is from 590 nm to 650 nm, wherein the light from the optical emitter propagates at a depth of 1 cm to 5 cm into the tissue as measured from a surface of the optical temperature monitoring device, detecting incident light with the optical detector, and determining the temperature of the tissue from the light detected by the optical detector.

In a twenty-fourth aspect, in addition to one or more of the preceding or following aspects, or in the alternative to some aspects, the method can further include implanting the optical temperature monitoring device in the patient's tissue.

In a twenty-fifth aspect, in addition to one or more of the preceding or following aspects, or in the alternative to some aspects, the method can further include orienting the optical temperature monitoring device such that optical emitter and the optical detector face internally relative to the skin of the patient.

In a twenty-sixth aspect, in addition to one or more of the preceding or following aspects, or in the alternative to some aspects, the method can further include emitting light into the tissue of the patient using a second optical emitter at a second wavelength.

In a twenty-seventh aspect, in addition to one or more of the preceding or following aspects, or in the alternative to some aspects, the optical emitter is disposed from 1 cm to 10 cm away from the optical detector.

In a twenty-eighth aspect, in addition to one or more of the preceding or following aspects, or in the alternative to some aspects, the method can further include measuring at least one of heart rate, respiration rate, circadian rhythm, and posture.

In a twenty-ninth aspect, in addition to one or more of the preceding or following aspects, or in the alternative to some aspects, the method can further include delivering a therapy to the patient.

In a thirtieth aspect, in addition to one or more of the preceding or following aspects, or in the alternative to some aspects, the method can further include measuring at least one of absorption, light scattering, and phase with the optical detector.

In a thirty-first aspect, an optical temperature monitoring system is included having a housing, and an optical temperature monitoring device, the optical temperature monitoring device can include an optical emission assembly, the optical emission assembly can include a first optical emitter configured to emit light at a first wavelength, wherein the first wavelength is from 590 nm to 650 nm, and a second optical emitter configured to emit light at a second wavelength, an optical detector, wherein the optical detector is configured to detect incident light, wherein the incident light detected by the optical detector is used to determine a temperature of the tissue, wherein the light from the optical emission assembly propagates through tissue, and wherein the optical temperature monitoring system is configured to be implanted in a subcutaneous tissue of a patient.

In a thirty-second aspect, in addition to one or more of the preceding or following aspects, or in the alternative to some aspects, wherein at least one of absorption, scattering and phase of the light detected by the optical detector is used to determine the temperature of the tissue.

In a thirty-third aspect, in addition to one or more of the preceding or following aspects, or in the alternative to some aspects, the absorption is used to determine an optical density of the tissue and the optical density is used to determine a temperature of the tissue.

In a thirty-fourth aspect, in addition to one or more of the preceding or following aspects, or in the alternative to some aspects, the temperature of the tissue is a function of the ratio of the detected light originating from the first optical emitter and the detected light originating from the second optical emitter.

In a thirty-fifth aspect, in addition to one or more of the preceding or following aspects, or in the alternative to some aspects, wherein both an AC component and an DC component of the light detected by the optical detector are used to determine the temperature of the tissue.

In a thirty-sixth aspect, in addition to one or more of the preceding or following aspects, or in the alternative to some aspects, the optical emitter and the optical detector are disposed on a substantially planar surface.

In a thirty-seventh aspect, in addition to one or more of the preceding or following aspects, or in the alternative to some aspects, the optical emitter is disposed 1 cm to 10 cm away from the optical detector.

In a thirty-eighth aspect, in addition to one or more of the preceding or following aspects, or in the alternative to some aspects, the optical temperature monitoring device of claim 1, wherein the light from the optical emitter propagates a depth of 1 cm to 5 cm into the tissue as measured from a surface of the optical temperature monitoring device.

In a thirty-ninth aspect, in addition to one or more of the preceding or following aspects, or in the alternative to some aspects, further can include at least one of a pulse oximetry sensor, chemical sensor, posture sensor, and heart rate sensor.

In a fortieth aspect, in addition to one or more of the preceding or following aspects, or in the alternative to some aspects, the temperature of the tissue is interpreted in view of at least one of heart rate, respiration, circadian rhythm, and posture.

In a forty-first aspect, in addition to one or more of the preceding or following aspects, or in the alternative to some aspects, wherein at least one of the optical emitter and the optical detector are disposed on a flexible member extending from the housing.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of an implanted optical temperature monitoring device in accordance with the embodiments herein.

FIG. 2 is a schematic view of an implanted optical temperature monitoring device system in accordance with the embodiments herein.

FIG. 3 is a schematic view of a wearable optical temperature monitoring device in accordance with the embodiments herein.

FIG. 4 is a schematic top view of a wearable optical temperature monitoring device system in accordance with the embodiments herein.

FIG. 5 is a schematic view of a system including implanted optical temperature monitoring devices in accordance with the embodiments herein.

FIG. 6 is a schematic view of an implanted optical temperature monitoring device in accordance with the embodiments herein.

FIG. 7 is a schematic view of an implanted optical temperature monitoring device system in accordance with the embodiments herein.

FIG. 8 is a schematic view of a wearable optical temperature monitoring device in accordance with the embodiments herein.

FIG. 9 is a schematic top view of a wearable optical temperature monitoring device system in accordance with the embodiments herein.

FIG. 10 is a schematic view of a system including implanted optical temperature monitoring devices in accordance with the embodiments herein.

FIG. 11 is a schematic top view of an optical temperature monitoring device in accordance with various embodiments herein.

FIG. 12 is a schematic top view of an optical temperature monitoring device in accordance with various embodiments herein.

FIG. 13 is a schematic top view of an optical temperature monitoring device in accordance with various embodiments herein.

FIG. 14 is a schematic top view of an optical temperature monitoring device in accordance with various embodiments herein.

FIG. 15 is a schematic cross-sectional view of an optical temperature monitoring device along line 15-15′ of FIG. 14 in accordance with various embodiments herein.

FIG. 16 is a schematic top view of an embodiment of an optical temperature monitoring device in accordance with various embodiments herein.

FIG. 17 is a schematic top view of an embodiment of an optical temperature monitoring device in accordance with various embodiments herein.

FIG. 18 is a schematic cross-sectional view of an implantable medical device in accordance with various embodiments herein.

FIG. 19 is a schematic top view of an embodiment of an optical temperature monitoring device in accordance with various embodiments herein.

FIG. 20 is a schematic top view of an embodiment of an optical temperature monitoring device in accordance with various embodiments herein.

FIG. 21 is a schematic top view of an embodiment of an optical temperature monitoring device in accordance with various embodiments herein.

FIG. 22 is a schematic cross-sectional view of an implanted optical temperature monitoring device in accordance with various embodiments herein.

FIG. 23 is a representative plot of tissue penetration depth of light at various tissue depths in accordance with various embodiments herein.

FIG. 24 is a schematic cross-sectional view of an implanted optical temperature monitoring device in accordance with various embodiments herein.

FIG. 25 is a schematic cross-sectional view of a wearable optical temperature monitoring device in accordance with various embodiments herein.

FIG. 26 is a schematic cross-sectional view of a wearable optical temperature monitoring device in accordance with various embodiments herein.

FIG. 27 is a representative plot of optical density temperature coefficient vs. wavelength.

FIG. 28 is a representative plot of temperature vs. energy in accordance with various embodiments herein.

FIG. 29 is a schematic representation oxygenation status versus time in accordance with various embodiments herein.

FIG. 30 is a schematic diagram of components of an optical temperature monitoring device in accordance with various embodiments herein.

FIG. 31 is a flow diagram of a method in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Embodiments herein relate to devices and methods for assessing deep tissue temperature using optical sensing. More specifically, embodiments herein relate to devices and methods for assessing temperature in deep tissue. As described above, peripheral monitoring of body temperature can be limited due to various factors and is limited to shallow tissue depths of under one centimeter (cm). The systems, devices, and methods herein are directed to determining and assessing temperature using optical sensing that can penetrate greater than one centimeter into the tissue.

In accordance with embodiments herein, tissue temperature can be determined from the optical properties of the tissue. Tissue contains various chromophores including, for example, the chromophores in blood related to oxygenated hemoglobin and deoxygenated hemoglobin. Chromophore optical density can be a function of temperature. Therefore, tissue temperature can be assessed by measuring the optical density of the tissue and converting the optical density measurements to a temperature measurement using the optical density temperature coefficient of tissue and/or components therein including blood.

In accordance with embodiments herein, light can be emitted from an optical emitter and can propagate deeply through a patient's tissues and back to an optical detector. As light travels through the tissues of a patient, various optical properties can be affected including transmittance, reflectance, absorption, light attenuation, scattering, wavelength, current, and/or fluorescence. Devices and systems herein can be configured to utilize the optical properties of light having passed through deep tissue to determine physiological parameters such as core temperature. Temperature can be measured at significant depths, such as at depths of greater than one centimeter. The devices herein can be configured to cause light to penetrate deeply into the tissue by optimizing emitter-detector spacing, wavelength selection, and/or using multiple wavelengths.

Devices and/or systems herein can take the form of implantable devices, wearable devices, or a combination of both. In various embodiments, the devices or systems herein can specifically include optical temperature monitoring devices that include implantable optical temperature monitoring devices, wearable optical temperature monitoring devices, or a combination of both types of devices. Referring now to FIG. 1, a schematic view of an implantable optical temperature monitoring device is shown in accordance with various embodiments herein. The optical temperature monitoring device 102 is shown positioned between the left fifth rib 104 and the left sixth rib 106 within the left fifth intercostal space of patient 100. While placement of the optical temperature monitoring device 102 is shown within the left fifth intercostal space, it will be appreciated that the optical temperature monitoring device 102 can be implanted within other locations of the body and, in particular, in or adjacent to other intercostal spaces along either the right side or the left side of a patient's rib cage or in other parts of the chest or torso.

The implantation depth of the optical temperature monitoring device can be from 1 millimeter (mm) to 25 mm or more below the skin layer of a patient's body. In some embodiments, the implantation depth below the skin layer can be greater than or equal to 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, or 30 mm, or can be an amount falling within a range between any of the foregoing.

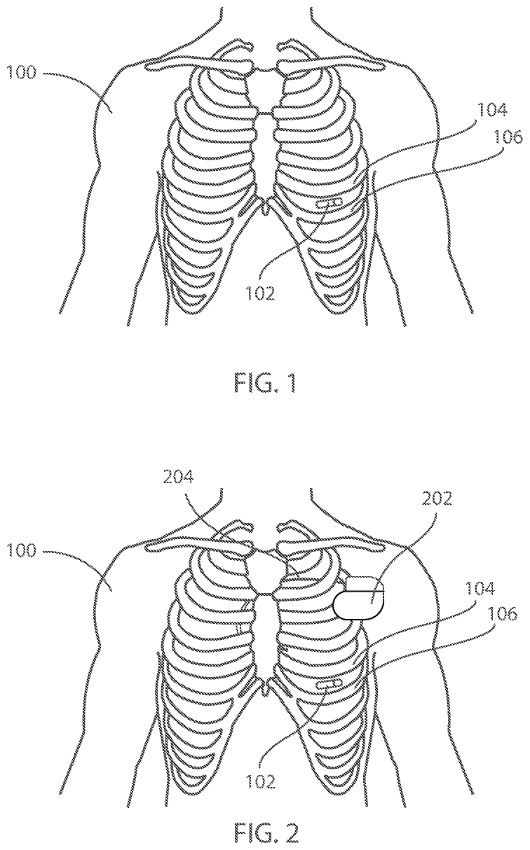

In some scenarios, the implantable optical temperature monitoring devices embodied herein can be used alone. However, in some scenarios, the implantable optical temperature monitoring devices embodied herein can be used in combination with or included within various types of implantable or non-implantable medical devices, including, but not limited to, implantable monitoring devices, such as implantable cardiac monitors and/or implantable therapeutic devices such as implantable cardiac rhythm management devices, implantable pacemakers, implantable cardioverter-defibrillator devices. The implantable monitoring devices and/or implantable therapeutic devices can be implanted along with the optical temperature monitoring devices described herein. Referring now to FIG. 2, a schematic view of an implantable optical temperature monitoring device is shown in accordance with various embodiments herein. The optical temperature monitoring device 102 is shown positioned between the left fifth rib 104 and the left sixth rib 106 within the left fifth intercostal space of patient 100. FIG. 2 further includes an implantable therapeutic device 202 positioned within the chest of the patient 100. Implantable therapeutic device 202 can be implanted at any position within patient 100 to provide a desired therapy to the patient in conjunction with signals determined by the optical temperature monitoring device, as will be discussed elsewhere herein. Implantable therapeutic device 202 can include one or more electrical stimulation leads 204 placed within the body of patient 100 at or near a treatment site.

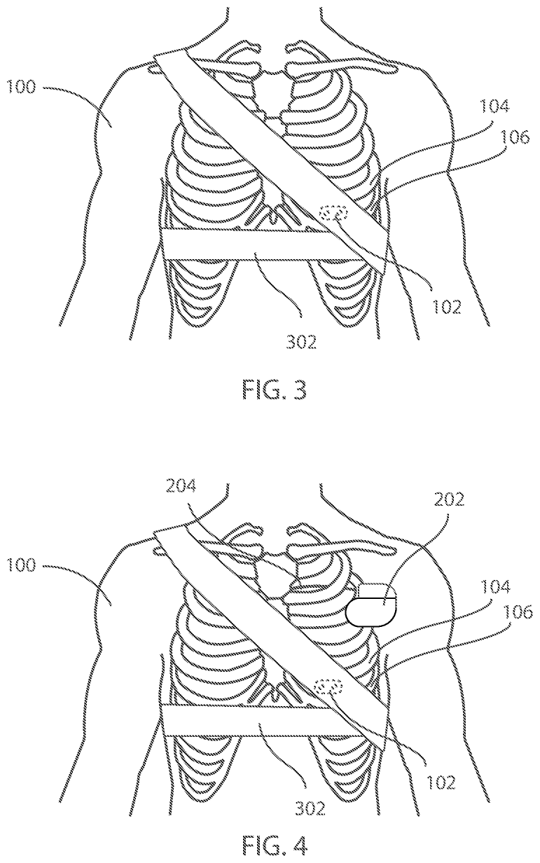

Optical temperature monitoring devices herein can specifically include wearable devices. Referring now to FIG. 3, a schematic view of a wearable optical temperature monitoring device is shown in accordance with various embodiments herein. The optical temperature monitoring device 102 can be disposed along a length of a wearable strap 302 and externally positioned between the left fifth rib 104 and the left sixth rib 106 over the left fifth intercostal space of patient 100. While placement of the optical temperature monitoring device 102 is shown placed over the left fifth intercostal space, it will be appreciated that the optical temperature monitoring device 102 can also be positioned over other intercostal spaces along either the right side or the left side of a patient's rib cage or at other sites on the skin of the patient. It will be appreciated that the wearable optical temperature monitoring devices herein can be in direct contact with a patient's skin, or can be in direct contact with a protective barrier, gel, or film that is placed in direct contact with a patient's skin.

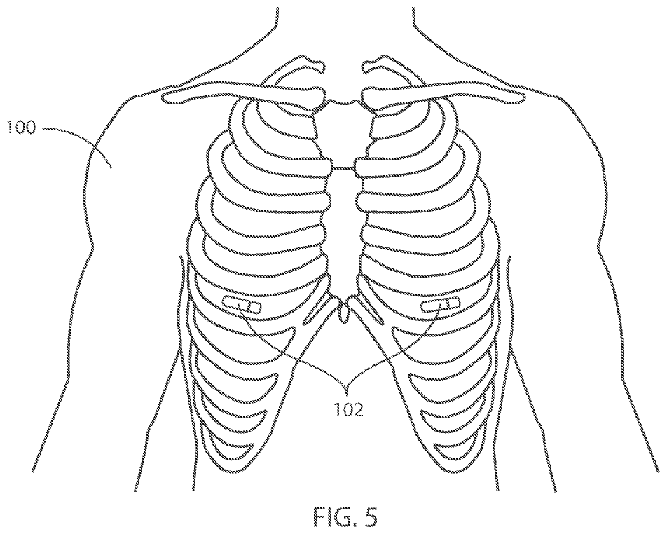

Even where optical temperature monitoring devices herein are external (such as wearable) they can still be used in combination with various types of implantable monitoring devices and/or implantable therapeutic devices, such as those described above. Thus, the implantable monitoring devices and/or implantable therapeutic devices can be implanted and used in a system in conjunction with the wearable optical temperature monitoring devices described herein. Referring now to FIG. 4, a schematic view of a wearable optical temperature monitoring device 102 is shown in accordance with various embodiments herein. The wearable optical temperature monitoring device 102 can be disposed along a length of a wearable strap 302 and externally positioned between the left fifth rib 104 and the left sixth rib 106 over the left fifth intercostal space of patient 100 or at another site. FIG. 4 further shows an implantable therapeutic device 202 positioned within the chest of the patient 100. Implantable therapeutic device 202 can be implanted at any position within patient 100 to provide a desired therapy to the patient in conjunction with signals determined by the optical temperature monitoring device, as discussed elsewhere herein. Implantable therapeutic device 202 can include one or more electrical stimulation leads 204 placed within the body of patient 100 at or near a treatment site. In some embodiments, the wearable temperature monitoring devices herein can be or include a patch sensor temporarily affixed to a patient by an adhesive, or can be in or on a garment or strap worn on the body of a patient.

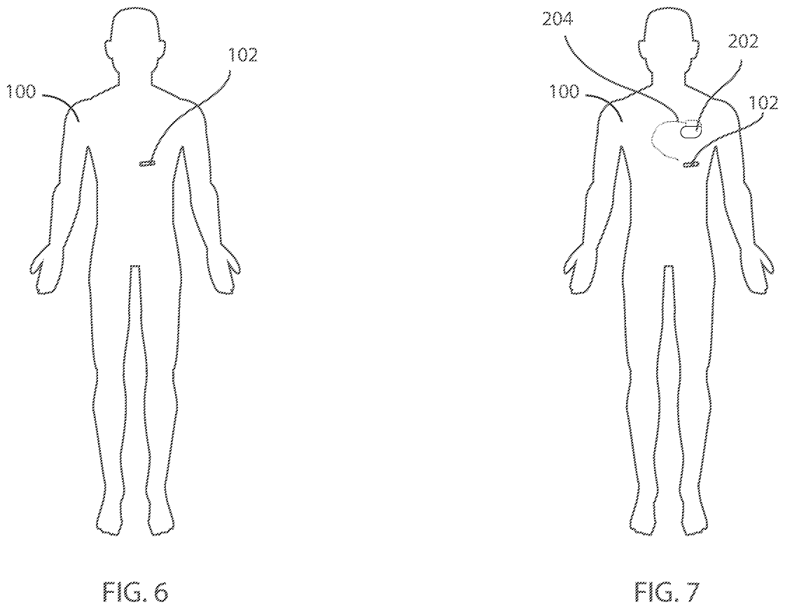

In some embodiments only a single optical temperature monitoring device may be implanted while in other embodiments multiple optical temperature monitoring devices can be implanted within a patient. Referring now to FIG. 5, a schematic view of a system with multiple implantable optical temperature monitoring devices is shown in accordance with various embodiments herein. The optical temperature monitoring devices 102 are shown in FIG. 5 as being positioned between the fifth rib and the sixth rib within the left fifth intercostal space and right firth intercostal space, respectively, but could also be placed at other sites in the body. While FIG. 5 specifically shows two optical temperature monitoring devices implanted within the patient 100, it will be appreciated that more than two optical temperature monitoring devices could be implanted. In some embodiments, three, four, five, or more optical temperature monitoring devices can be implanted within patient 100. Furthermore, it will be appreciated that a combination of two or more optical temperature monitoring devices can be worn on an exterior of a patient's body.

In various embodiments, optical temperature monitoring devices can be implanted along with at least one secondary sensor. The secondary sensors can include, but are not to be limited to, a pulse oximetry sensor, a chemical sensor, a posture sensor, or a heart rate sensor. Secondary sensors can be used to determine one or more of a heart rate, a respiratory rate, a tidal volume, or an extravascular lung water concentration. The secondary sensor(s) can be a part of the optical temperature monitoring devices or can be separate.

It will be appreciated that the placement of the optical temperature monitoring device is not restricted to an intercostal space as depicted in FIGS. 1-5. The optical temperature monitoring device can be placed anywhere in the patient's body where light can propagate a sufficient depth into the body of a patient to measure a temperature of a patient's deep tissue. In various embodiments, the optical temperature monitoring device can be placed anywhere in the chest, abdomen, torso, neck, head, or limbs of a patient.

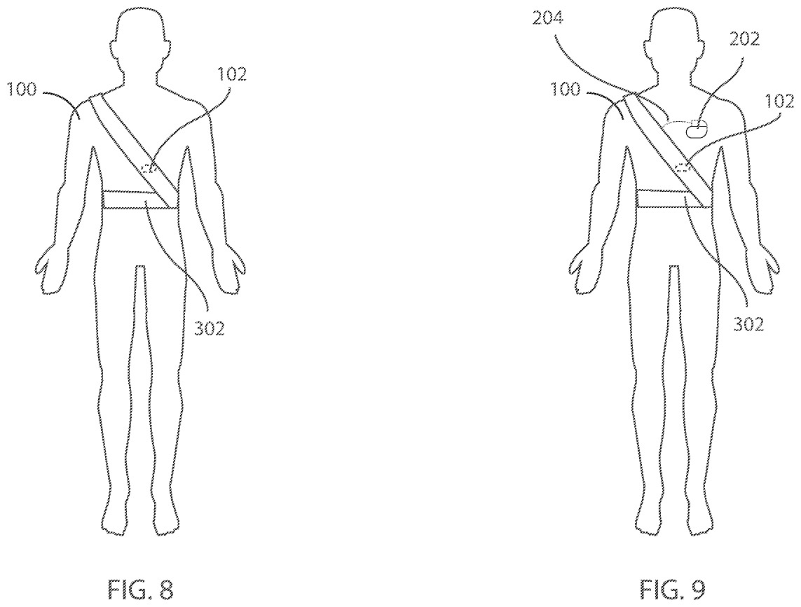

Referring now to FIG. 6, a schematic view of an optical temperature monitoring device is shown in accordance with various embodiments herein. The optical temperature monitoring device 102 is shown in FIG. 6 as being positioned in the chest area of the patient 100, but not within an intercostal space. In the configuration of FIG. 6, the optical temperature monitoring device 102 can measure a core temperature of the patient 100. Similarly, referring now to FIG. 7, a schematic view of an optical temperature monitoring device is shown in accordance with various embodiments herein. The optical temperature monitoring device 102 is shown in FIG. 7 as being positioned in the chest of a patient 100, but not within an intercostal space. FIG. 7 further includes an implantable therapeutic device 202 positioned within the chest of the patient 100 along with one or more electrical stimulation leads 204.

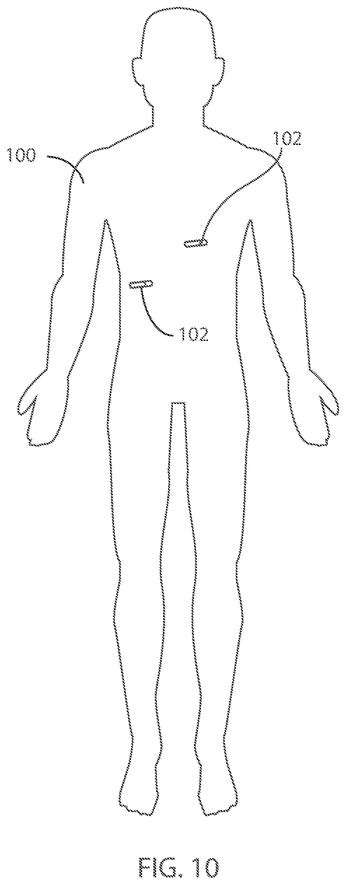

The optical temperature monitoring devices herein can include those that are wearable devices that can be temporarily held against or affixed to the skin in a desired area of the body including, but not limited to, somewhere on the chest, neck, torso or other part of a patient. The optical temperature monitoring device can be a patch sensor temporarily affixed to a patient by an adhesive, or it can be held against the skin using a garment or strap worn on the body of a patient. Referring now to FIG. 8, a schematic view of a wearable optical temperature monitoring device is shown in accordance with various embodiments herein. The optical temperature monitoring device 102 can be disposed along a length of a wearable strap 302 and externally on the chest of the patient 100. While placement of the optical temperature monitoring device 102 is shown placed over a patient's chest, it will be appreciated that the optical temperature monitoring device can be placed anywhere on a patient's body where light can propagate a sufficient depth into the body to measure a temperature of the patient's deep tissue. In various embodiments, the optical temperature monitoring device can be placed on a patient's chest, back, abdomen, head, or limbs. As discussed before, the wearable optical temperature monitoring devices embodied herein can be used in combination with various types of implantable therapeutic devices. Referring now to FIG. 9, a schematic view of a wearable optical temperature monitoring device 102 is shown in accordance with various embodiments herein. The wearable optical temperature monitoring device 102 can be disposed along a length of a wearable strap 302 and externally positioned on the body of patient 100. FIG. 9 further shows an implantable therapeutic device 202 along with one or more electrical stimulation leads 204 positioned within the chest of the patient 100, but can be implanted at any position within patient 100 to provide a desired therapy to the patient in conjunction with signals determined by the optical temperature monitoring device, as will be discussed elsewhere herein. Wireless signals can be exchanged between the wearable optical temperature monitoring device 102 and the implantable therapeutic device 202.

Multiple optical temperature monitoring devices can be implanted within a patient in conjunction with the embodiments herein. Referring now to FIG. 10, a schematic view of a system with multiple implantable optical temperature monitoring devices is shown in accordance with various embodiments herein. The optical temperature monitoring devices 102 are shown in FIG. 10 as being positioned within the chest of a patient at different locations.

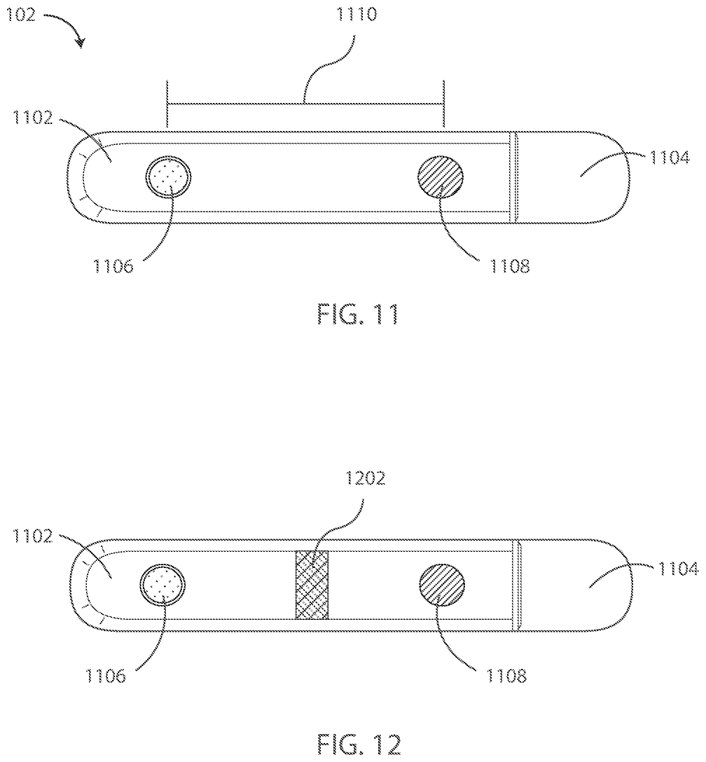

The optical temperature monitoring devices herein can include various components, such as optical emitters, optical detectors, secondary sensors, and optical barriers in various configurations in accordance with the embodiments herein and as shown and described in reference to FIGS. 11-21. In some embodiments, the optical emitters and optical detectors can be disposed together on the same side of the optical temperature monitoring device while the secondary sensors can be disposed on the opposite side of the optical temperature monitoring device. However, in other embodiments, the optical emitters, optical detectors, and secondary sensors can all be disposed together on the same side of the optical temperature monitoring device.

Referring now to FIG. 11, an optical temperature monitoring device 102 is shown in accordance with various embodiments herein. In this embodiment, the optical temperature monitoring device 102 can include a housing 1102 and a header 1104 coupled to the housing 1102. Various materials can be used to form the housing 1102 and the header 1104. In some embodiments, the housing 1102 can be formed of a material such as a metal, ceramic, a polymer, or a composite. The header 1104 can be formed of various materials, and in some embodiments the header 1104 can be formed of a translucent polymer such as an epoxy material. In some embodiments the header 1104 can be hollow. In other embodiments the header 1104 can be filled with components and/or structural materials such as epoxy or another material such that it is non-hollow. In yet other embodiments, the optical temperature monitoring device 102 can be devoid of a header 1104 or can include a header at either end or both ends of the optical temperature monitoring device. In an embodiment, part of all of housing 1102 can be transparent to provide a window for optical components of oxygenation monitoring device 102.

In various embodiments, the optical temperature monitoring device 102 can include a first optical emitter 1106 and a first optical detector 1108, each coupled to the housing 1102. The first optical emitter 1106 can be configured to emit light in the UV, visible, or near-infrared spectra. The first optical emitter 1106 can be configured to emit light at a first wavelength of from 100 nanometers (nm) to 2000 nm. In various embodiments, the first optical emitter can be configured to emit light at a first wavelength of from 590 nm to 650 nm. In some embodiments, the first optical emitter can be configured to emit light at a first wavelength of from 605 nm to 615 nm. In various embodiments, the first optical emitter 1106 can be configured to emit light at a first wavelength that can be greater than or equal to 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, 1600 nm, 1700 nm, 1800 nm, 1900 nm, or 2000 nm, or can be any wavelength falling within a range between any of the foregoing. The light emitted by the optical emitter can propagate through the tissue of a patent.

The first optical detector 1108 can be configured to detect incident light originating from one or more optical emitters, such as the first optical emitter 1106. The first optical detector 1108 can be configured to detect light after it has propagated into the tissue of a patient to a given depth from the first optical emitter 1106 and back to the optical detector 1108. For example, the optical temperature monitoring device 102 can be configured to provide for the propagation of the emitted light through a tissue such that propagation of the emitted light occurs from about 1 centimeter (cm) in depth to 5 cm in depth as measured from a surface of the optical temperature monitoring device 102 to a target tissue. In some embodiments, the depth or propagation of the emitter light can be greater than or equal to 1 cm, 2 cm, 3 cm, 4 cm, or 5 cm, or more, or can be an amount falling within a range between any of the foregoing. In various embodiments, the configuration of the optical temperature monitoring device 102 can allow for the propagation of the emitted light through a tissue when a surface of the optical temperature monitoring device 102 is directed to the interior of a patient's body toward the surface of the deep tissue. The detected incident light can be used to determine a temperature of a tissue within a patient, as will be discussed elsewhere herein.

It will be appreciated that first optical emitter 1106 and first optical detector 1108 can be positioned at any location along temperature monitoring device 102 to achieve optimal propagation of light into the tissue of a patient. The first optical emitter 1106 and first optical detector 1108 can be spaced along a length of the optical temperature monitoring device 102 at a distance 1110 from 1 cm to 10 cm apart. In some embodiments, the distance 1110 between the first optical emitter and the first optical detector can be greater than or equal to 1 cm, 2 cm, 3 cm, 4 cm, 5 cm, 6 cm, 7 cm, 8 cm, 9 cm, 10 cm, 11 cm, 12 cm, 13 cm, 14 cm, or 15 cm, or can be an amount falling within a range between any of the foregoing. In various embodiments, the first optical emitter and the first optical detector are specifically disposed along a planar surface of the optical temperature monitoring device from 1 cm to 10 cm apart. Subject to other factors, the greater distance 610 is, the greater the depth of propagation of light through the tissue.

The optical temperature monitoring device 102 can take on various dimensions in length, width, and thickness. In a particular embodiment herein, it can be approximately 5 cm to 10 cm in length, 1 cm to 1.5 cm wide, and 0.25 cm to 1.0 cm thick. In some embodiments, the length of temperature monitoring device 102 can be greater than or equal to 1 cm, 2 cm, 3 cm, 4 cm, 5 cm, 6 cm, 7 cm, 8 cm, 9 cm, 10 cm, 11 cm, 12 cm, 13 cm, 14 cm, or 15 cm, or can be an amount falling within a range between any of the foregoing. In some embodiments, the optical temperature monitoring device 102 can be about 0.25 cm, 0.5 cm, 0.75 cm, 1.0 cm, or 2.0 cm in width. In some embodiments the width can be in a range wherein any of the foregoing widths can serve as the upper or lower bound of the range, provided that the upper bound is greater than the lower bound. In some embodiments, the optical temperature monitoring device 102 can be about 0.25 cm, 0.50 cm, 0.75 cm or 1.0 cm, 1.25 cm, 1.50 cm, 1.75 cm, 2.0 cm, 2.25 cm, 2.50 cm, or 3.0 cm thick, or can be an amount falling within a range between any of the foregoing. In some embodiments the thickness can be in a range wherein any of the foregoing thicknesses can serve as the upper or lower bound of the range, provided that the upper bound is greater than the lower bound.

In some embodiments, the optical temperature monitoring devices herein can include an optical barrier device disposed between the optical emitters and optical detectors to prevent a direct path for light emitted from an optical emitter to an optical detector. Referring now to FIG. 12, an optical temperature monitoring device 102 is shown in accordance with the embodiments herein. The optical temperature monitoring device 102 can include the features of the optical temperature monitoring device 102 of FIG. 11 and can further include an optical barrier device 1202 disposed in between the first optical emitter 1106 and the first optical detector 1108. In some embodiments, the optical barrier device 1202 can be mounted on the surface of the temperature monitoring device 102. In some embodiments, the optical barrier device 1202 can be embedded in the surface of the temperature monitoring device 102. The optical barrier device 1202 can include various materials including light blocking materials such as opaque compositions and materials, polymers, metals, dyed materials, and the like.

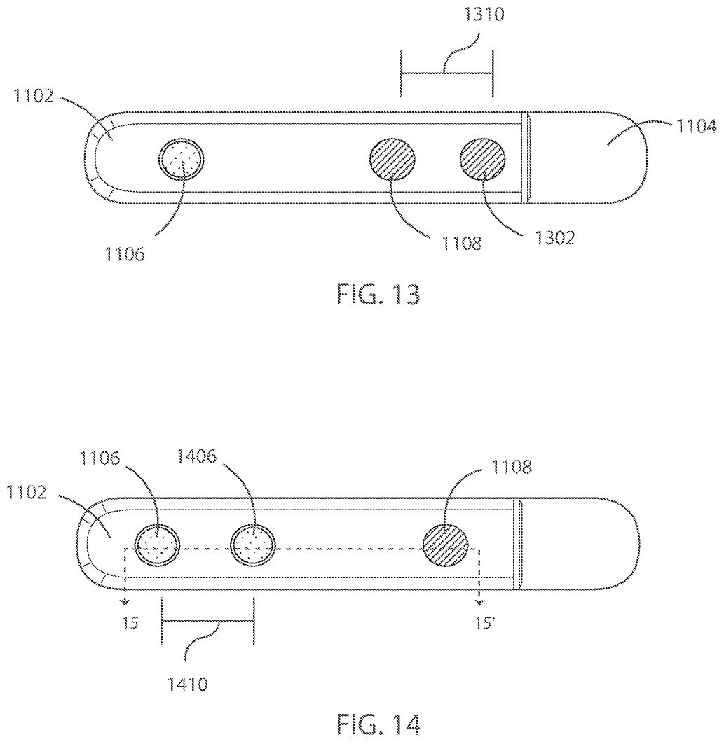

It will be appreciated that the optical temperature monitoring devices embodied herein can include various configurations of multiple optical emitters or optical detectors disposed along a length of the optical temperature monitoring devices. Referring now to FIG. 13, an optical temperature monitoring device 102 is shown in accordance with the embodiments herein. The optical temperature monitoring device 102 can include a housing 1102 and a header 1104 coupled to the housing 1102. The optical temperature monitoring device 102 can include a first optical emitter 1106, a first optical detector 1108, and a second optical detector 1302, each coupled to the housing 1102. The first optical emitter 1106 and first optical detector 1108 can be spaced along a length of the optical temperature monitoring device 102 as discussed in reference to FIG. 11. The first optical detector 1108 and the second optical detector 1302 can be disposed along a length of the optical temperature monitoring device 102 at a distance 1310 of from 1 cm to 5 cm or more apart. The second optical detector 1302 can be configured to detect incident light. In some embodiments, the distance 1110 between the first optical detector and the second optical detector can be greater than or equal to 0.25 cm, 0.5 cm, 1 cm, 2 cm, 3 cm, 4 cm, or 5 cm, or can be an amount falling within a range between any of the foregoing.

Referring now to FIG. 14, an optical temperature monitoring device 102 is shown in accordance with the embodiments herein. The optical temperature monitoring device 102 can include a housing 1102 and a header 1104 coupled to the housing 1102. The optical temperature monitoring device 102 can include a first optical emitter 1106, a second optical emitter 1406, and first optical detector 1108, each coupled to the housing 1102. The first optical emitter 1106 and first optical detector 1108 can be spaced along a length of the optical temperature monitoring device 102 as discussed in reference to FIG. 11. The first optical emitter 1106 and the second optical emitter 1406 can be disposed along a length of the optical temperature monitoring device 102 at a distance 1410 of from 1 cm to 5 cm or more apart. In some embodiments, the distance 1410 between the first optical emitter and the second optical emitter can be greater than or equal to 0.25 cm, 0.5 cm, 1 cm, 2 cm, 3 cm, 4 cm, or 5 cm, or more, or can be an amount falling within a range between any of the foregoing. It will be appreciated, however, that the depth of propagation of light through tissue is dependent on the distance between the emitter and the detector and not the distance between the emitters.

The second optical emitter 1406 can be configured to emit light at a second wavelength when present on an optical temperature monitoring device with a first optical emitter. In various embodiments, the second optical emitter 1406 can be configured to emit light at a second wavelength different than the first wavelength emitted from the first optical emitter 1106. In some embodiments, the second optical emitter can be configured to emit light at a second wavelength of from 750 nm to 1500 nm. In various embodiments, the first optical emitter 1106 can be configured to emit light at a second wavelength that can be greater than or equal to 700 nm, 750 nm, 800 nm, 900 nm, 1000 nm, 1100 nm, 1200 nm, 1300 nm, 1400 nm, 1500 nm, 1600 nm, 1700 nm, 1800 nm, 1900 nm, or 2000 nm, or can be an amount falling within a range between any of the foregoing. In various embodiments, the second wavelength is a near-infrared wavelength.

It will be appreciated that optical emitters herein can be used substantially continuously or only during certain time periods. For example, in some cases, the device and/or system can be configured to measure temperature continuously or substantially continuously. In such a scenario, an optical emitter can be turned on continuously or intermittently as part of a duty cycle such as a certain fraction of time that the emitter is emitting light. It will be appreciated, however, that continuous operation of an emitter may consume substantial energy and lower the battery life of an implanted device. Thus, as a further example, in some embodiments, the device or system can evaluate temperature only during certain periods of time. For example, the device or system can evaluate temperature only when it receives a command to measure temperature coming from a different device or from a clinician or other system user. As another example, the device or system can evaluate temperature according to a preset schedule. As another example, the device or system can evaluate temperature after detecting a particular occurrence or event using one or more sensors, such as an abnormal heart rhythm, an abnormal respiration pattern, or the like. In scenarios where there is more than one emitter, the emitters can be turned on simultaneously or in an alternating pattern.

It will be appreciated that the optical emitters and optical detectors can be formed from various materials and/or be of various types. Optical emitters can include a light source such as a light emitting diode (LED), vertical-cavity surface-emitting lasers (VCSELs), electroluminescent (EL) devices, and the like. Optical detectors can include a component selected from the group including a photodiode, a phototransistor, a charge-coupled device (CCD), a junction field effect transistor (JFET) optical sensor, a complementary metal-oxide semiconductor (CMOS) optical sensor, an integrated photo detector integrated circuit, a light to voltage converter, and the like. Optical emitters and optical detectors are discussed in further detail below.

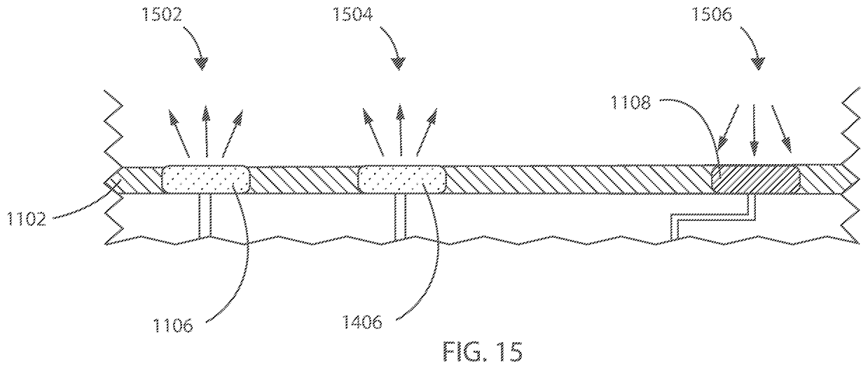

Referring now to FIG. 15, a cross-sectional view of an optical temperature monitoring device is shown in accordance with various embodiments herein. Optical temperature monitoring device 102 includes housing 1102 having a first optical emitter 1106, a second optical emitter 1406, and a first optical detector 1108. The first optical emitter 1106 can be configured to emit light at a first wavelength 1502 from 100 nm to 2000 nm. The second optical emitter 1406 can be configured to emit light at a second wavelength 1504 from 800 nm to 2000 nm. In various embodiments, he emitted light of a first wavelength from the first optical emitter can be propagated to a different depth than the emitted light of a second wavelength from the second optical emitter. The first optical detector 1108 is configured to detect incident light 1506 that returns to the first optical detector 1108 from either the first optical emitter 1106, the second optical emitter 1406, or both. It will be appreciated that the optical temperature monitoring devices herein can include any combination of one or more optical emitters, optical detectors, and secondary sensors, as described below.

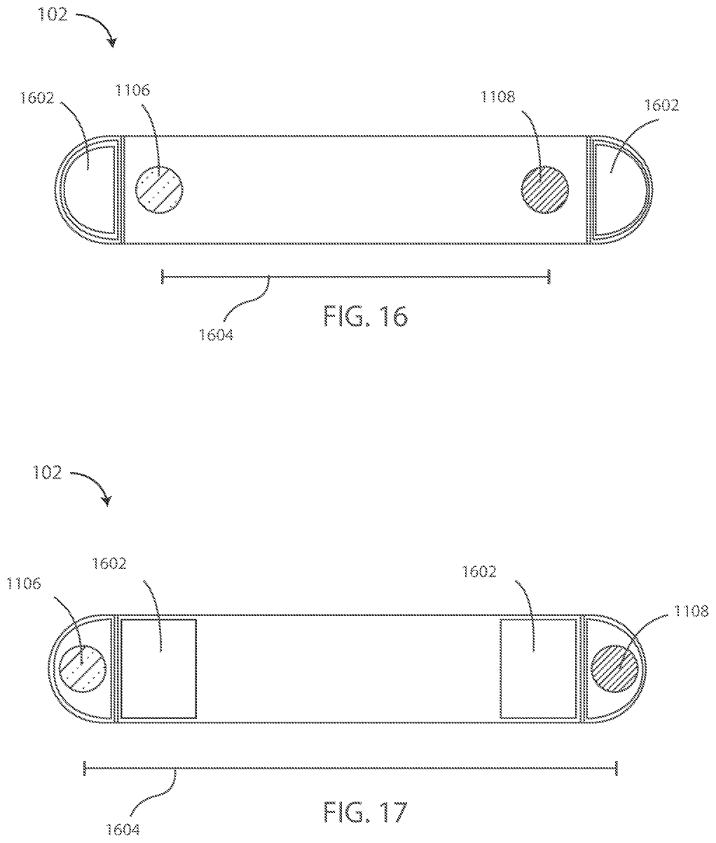

Referring now to FIGS. 16-17, schematic views of additional embodiments of the optical temperature monitoring devices are shown in accordance with various embodiments herein. The optical temperature monitoring devices 102 can include a first optical emitter 1106 and a first optical detector 1108. While only one optical emitter and one optical detector are shown in the optical temperature monitoring devices 102 of FIGS. 16-17, it will be appreciated that the optical temperature monitoring devices 102 can include more than one optical emitter, more than one optical detector, and a combination of optical emitters and optical detectors. The optical temperature monitoring devices 102 can further include one or more electrodes 1602 disposed along a length of the optical temperature monitoring devices 102. Electrodes 1602 can be configured to function as part of a secondary sensor (such as an ECG sensor) or, in some embodiments, deliver an electrical stimulation therapy to a patient at or near a treatment site, as will be discussed elsewhere herein.

The first optical emitter 1106 and the first optical detector 1108, as shown in FIGS. 16 and 17 can be disposed along a length of the optical temperature monitoring device separated by a predetermined distance 1604. Predetermined distance 1604 can include a distance such that the first optical emitter 1106 and the first optical detector 1108 are spaced along a length of the optical temperature monitoring device from 1 cm to 10 cm apart. In some embodiments, a predetermined distance 1604 between the first optical emitter and the first optical detector can be greater than or equal to 1 cm, 2 cm, 3 cm, 4 cm, 5 cm, 6 cm, 7 cm, 8 cm, 9 cm, 10 cm, 11 cm, 12 cm, 13 cm, 14 cm, or 15 cm, or can be an amount falling within a range between any of the foregoing.

In FIG. 16, the first optical emitter 1106 and the first optical detector 1108 are positioned between electrodes 1602. In some cases, this can be advantageous because depending on the overall dimensions of the device there may be limited space inside of the device in the areas at the ends of the device. However, in FIG. 17, the first optical emitter 1106 and the first optical detector 1108 are positioned outside of electrodes 1602. In some cases, this can be advantageous to achieve a maximal spacing between the emitter and detector for a given overall device size. This can be important because a significant factor in the depth of light propagation through the tissue is the distance between the emitter and the detector where a greater distance generally results in a greater depth of propagation through the tissue.

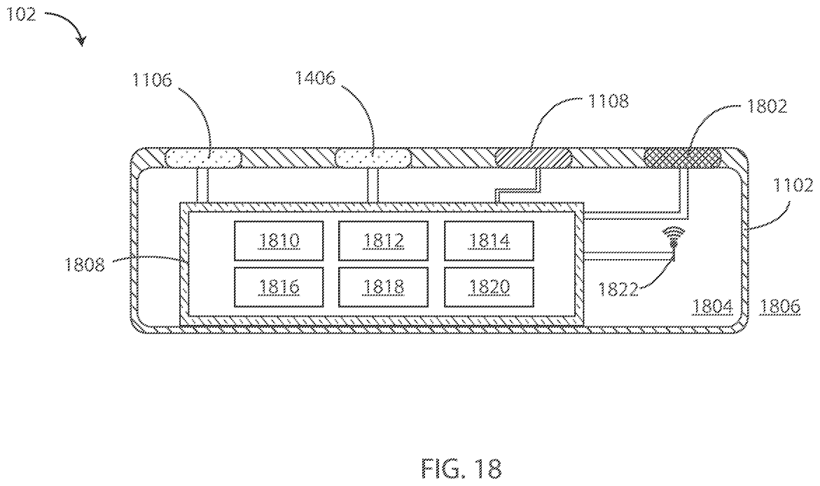

Referring now to FIG. 18, a schematic cross-sectional view of an optical temperature monitoring device 102 is shown in accordance with various embodiments herein. The optical temperature monitoring device 102 can include housing 1102. The housing 1102 of optical temperature monitoring device 102 can include various materials such as metals, polymers, ceramics, and the like. In some embodiments, all or part of housing 1102 can be transparent. In some embodiments, the housing 1102 can be a single integrated unit. In other embodiments, the housing 1102 can include housing 1102 and header, as discussed above. In some embodiments, the housing 1102, or one or more portions thereof, can be formed of a biocompatible metal, such as titanium. In some embodiments, one or more segments of the housing 1102 can be hermetically sealed. In some embodiments, all or part of the housing 1102 can be transparent.

Housing 1102 can define an interior volume 1804 that in some embodiments is hermetically sealed off from the area 1806 outside of optical temperature monitoring device 102. The optical temperature monitoring device 102 can include control circuitry 1808. Control circuitry 1808 can include various components, such as components 1810, 1812, 1814, 1816, 1818, and 1820. In some embodiments, some or all of these components can be integrated and in other embodiments these components can be separate. In some embodiments, the components can include one or more of a microprocessor, memory circuitry (such as random-access memory (RAM) and/or read only memory (ROM)), recorder circuitry, telemetry circuitry, measurement circuitry, chemical sensor interface circuitry, power supply circuitry (which can include one or more batteries), normalization circuitry, optical temperature monitoring device control circuitry, optical emitter control circuitry, optical detector control circuitry, and the like. In some embodiments, recorder circuitry can record the data produced by the optical temperature monitoring device and record time stamps regarding the same. In some embodiments, the circuitry can be hardwired to execute various functions, while in other embodiments the circuitry can be implemented as instructions executing on a microprocessor or other computation device. In various embodiments, optical temperature monitoring device further includes a measurement circuit configured to control operation of the first optical emitter and the first optical detector

A telemetry interface 1822 can be provided for communicating with external devices such as a programmer, a home-based unit, and/or a mobile unit (e.g., a cellular phone, portable computer, etc.) or a wearable medical device. In some embodiments telemetry interface 1822 can be provided for communicating with implanted devices such as a therapy delivery device (e.g., a pacemaker, cardioverter-defibrillator, or the like) or monitoring-only device (e.g., an implantable loop recorder). In some embodiments, the circuitry can be implemented remotely, via either near-field, far-field, conducted, intra-body or extracorporeal communication, from instructions executing on any of the external or the implanted devices, etc. In some embodiments, the telemetry interface 1822 can be located within housing 1102. In some embodiments, the telemetry interface 1822 can be located in header.

Various optical emitters, optical detectors, and other secondary sensors as described elsewhere herein can be in electrical communication with the circuitry. FIG. 13, shows a first optical emitter 1106, a second optical emitter 1406, a first optical detector 1108 and a secondary sensor 1802 in electrical communication with the control circuitry 1808 within the interior volume 1804. In some embodiments, the control circuitry 1808 is configured to selectively activate first optical emitter 1106, the second optical emitter 1406, the first optical detector 1108 and the secondary sensor 1802. It will be appreciated that the control circuitry 1808 can be configured to selectively activate any number of optical emitters, optical detectors, and secondary sensors.

The secondary sensor 1802 can include, but is not limited to, one or more of a pulse oximetry sensor, a chemical sensor, a posture sensor, or a heart rate sensor. The secondary sensor 1802 can be configured to use electrical, optical, pressure, acoustic, or other techniques. In one example, the secondary sensor 1802 can include one or more electrodes to detect electrical properties including, but not limited, to impedance, electrical potential such as in the case of an ECG signal, and the like. In another example, the secondary sensor 1802 can include a microphone or device to detect pressure waves or vibration such as an accelerometer to detect characteristics sounds associated with the heart, lungs, or other physiological activity. In another example, the secondary sensor 1802 can include an accelerometer to detect posture. Aspects of exemplary chemical sensors are described in U.S. Pat. Nos. 7,809,441 and 8,126,554, the content of which is herein incorporated by reference.

It will be appreciated that there can be advantages associated with spacing emitters and detectors as far apart as possible to achieve as deep as possible tissue propagation of the emitted light. Generally, a limit on maximum spacing is the overall dimensions of the device and/or portions thereof such as the housing. However, in some embodiments, to achieve greater spacing, another structure can be attached to the device and components such as the emitter and/or detector can be mounted thereon. In this way, a maximum distance of separation can be increased based on the length of the structure added. Such a structure can take various forms. In some embodiments, the structure can be substantially rigid while in other embodiments the structure can be flexible. While not intending to be bound by theory it can be advantageous to include a flexible structure because a long rigid device can be more likely to cause discomfort for the individual into which the device is implanted (in an implantable embodiment). Thus, in various embodiments, the optical temperature monitoring device can include a flexible member. Flexible members embodied herein can assume many shapes, sizes, and configurations suitable for placement within a patient. In some embodiments, the flexible member can be substantially hollow other than components disposed therein. In some embodiments, the flexible member can be non-hollow and filled with a material such as a polymer, a composite, or the like. In contrast, the housing 1102 can be substantially rigid. However, in other embodiments the housing 1102 itself can also be flexible.

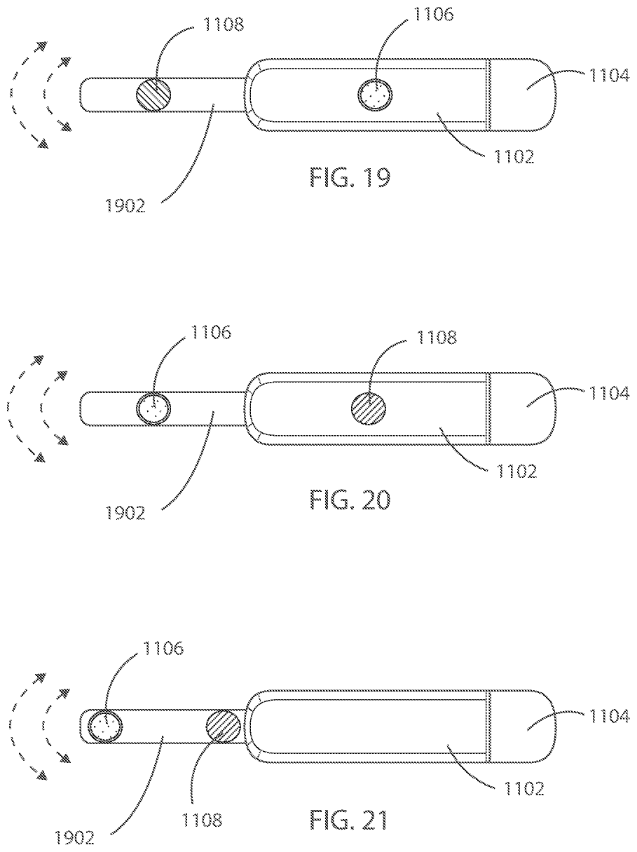

Referring now to FIGS. 19-21 schematic top plan views of an optical temperature monitoring device having a flexible member are shown according to various embodiments herein. Specifically, referring to FIG. 19, the optical temperature monitoring device 102 includes a housing 1102 and a header 1104 coupled to the housing 1102. The optical temperature monitoring device 102 can further include a flexible member 1902 extending from the housing 1102. In various embodiments, the flexible member 1902 can be connected to the housing and extend outward in a direction away from the housing 1102. As depicted in FIG. 19, the flexible member 1902 can extend outwards from the housing 1102 on the opposite side of the housing from the header 1104. Alternatively, the flexible member 1902 can extend outwards from the housing 1102 on the same side of the housing from the header 1104 and/or be an appendage of the header 604 itself. The flexible member 1902 can be constructed from various materials configured to be bent without damage. In some embodiments, the flexible member 1902 can be formed of a material such as a polymer, an elastomeric polymer, a flexible composite, or the like. In some embodiments, the flexible member 1902 can be of substantially the same diameter and/or circumference as the housing 1102, but in other embodiments can be greater or lesser (such as depicted in FIGS. 19-21) in diameter and/or circumference. While not shown in FIGS. 19-21, one or more conductors can interconnect the flexible member and components disposed thereon such as an emitter and/or detector with the other components of the device that may be housed in housing 1102.

It will be appreciated that various combinations of optical emitters and optical detectors can be used when a flexible member is present in the temperature monitoring devices herein. In various embodiments, the first optical emitter is disposed along a length of the flexible member, while the first optical detector is disposed along the housing. In various embodiments, the first optical detector is disposed along a length of the flexible member, while the first optical emitter is disposed along the housing. In other embodiments, the first optical emitter and the first optical detector are both disposed along a length of the flexible member. In some embodiments one or more optical detectors and one or more optical emitters can be used along with one or more secondary sensors that can be disposed along a temperature monitoring device having a flexible member.

The flexible member can take on various dimensions in the length, width, and thickness directions. In a particular embodiment herein, the flexible member can be approximately 1 to 5 cm in length, 0.25 cm to 1.5 cm wide, and 0.25 cm to 1.0 cm thick. In various embodiments, the flexible member is from 1 centimeter (cm) to 3 cm in length. In some embodiments, the length of flexible member 1902 can be greater than or equal to 1 cm, 2 cm, 3 cm, 4 cm, or 5 cm, or more or can be an amount falling within a range between any of the foregoing. In some embodiments, the flexible member 1902 can be about 0.25 cm, 0.5 cm, 0.75 cm, 1.0 cm, or 2.0 cm in width. In some embodiments the length can be in a range wherein any of the foregoing widths can serve as the upper or lower bound of the range, provided that the upper bound is greater than the lower bound. In some embodiments, the flexible member 1902 can be about 0.25 cm, 0.50 cm, 0.75 cm or 1.0 cm, 1.25 cm, 1.50 cm, 1.75 cm, or 2.0 cm thick, or can be an amount falling within a range between any of the foregoing. In some embodiments the thickness can be in a range wherein any of the foregoing thicknesses can serve as the upper or lower bound of the range, provided that the upper bound is greater than the lower bound. In some embodiments, the flexible member 1902 is sufficiently flexible that it can bend at an angle (with respect to the housing of optical temperature monitoring device) of at least 5, 10, 15, 20, 30, 45, 60 or 90 degrees or more.

In the embodiment of FIG. 19, the optical temperature monitoring device 102 can include a first optical emitter 1106 coupled to the housing and a first optical detector 1108 coupled to the flexible member 1902. However, it will be appreciated that the first optical emitter 1106 and the first optical detector 1108 can be positioned at any location along the optical temperature monitoring device 102, including the flexible member 1902 to achieve optimal propagation of light through the tissue of a patient.

Referring now to FIG. 20, an optical temperature monitoring device 102 is shown in accordance with the embodiments herein. The optical temperature monitoring device 102 can include a housing 1102, a header 1104 coupled to the housing 1102, and a flexible member 1902 extending from the housing 1102. In the embodiment of FIG. 20, the optical temperature monitoring device 102 can include a first optical emitter 1106 coupled to the flexible member 1902 and a first optical detector 1108 coupled to the housing 1102.

Referring now to FIG. 21, an optical temperature monitoring device 102 is shown in accordance with the embodiments herein. The optical temperature monitoring device 102 can include a housing 1102, a header 1104 coupled to the housing 1102, and a flexible member 1902 extending from the housing 1102. The optical temperature monitoring device 102 can include a first optical emitter 1106 and a first optical detector 1108 each coupled to the flexible member 1902.

While only one optical emitter and one optical detector are shown in the optical temperature monitoring devices 102 of FIGS. 19-21, it will be appreciated that the optical temperature monitoring devices 102 can include more than one optical emitter, more than one optical detector, and a combination of optical emitters and optical detectors disposed anywhere across the length of the optical temperature monitoring device.

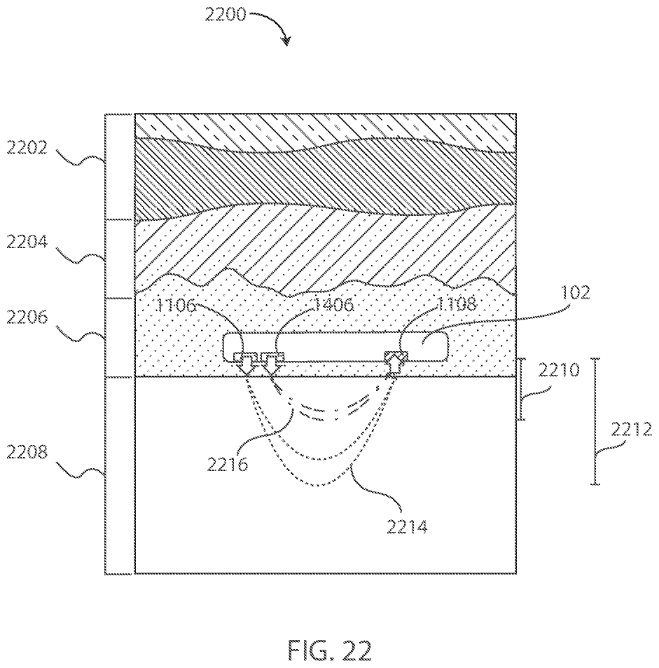

The temperature monitoring devices herein can be implanted within various subcutaneous implantation sites or other regions inside of the body. Referring now to FIG. 22, a schematic cross-sectional view of a subcutaneous implantation site 2200 with an implanted optical temperature monitoring device 102 is shown in accordance with various embodiments herein. Human skin includes multiple layers including the epidermis 2202 and the dermis 2204. Beneath the layers of human skin and typically above a layer of muscle 2208 is the subcutaneous space that can include a layer of adipose tissue 2206. It will be appreciated that the subcutaneous space further includes additional anatomical structures such as blood vessels, fascia, lymphatic vessels, nervous tissue, hair follicles, and the like.

The optical temperature monitoring device 102 can be disposed at any location within the subcutaneous space. In various embodiments, the optical temperature monitoring device 102 can even be implanted deeper within the body, such as within underlying muscle, within a cavity, such as the chest cavity, adjacent to or within an internal organ of the body, within a blood vessel, or the like. In various embodiments, the optical temperature monitoring device 102 can be held in place via sutures. In some embodiments, the optical temperature monitoring device 102 can include one or more apertures to facilitate its fixation via sutures. The optical temperature monitoring device 102 can be implanted within a patient such that each of the optical emitters and optical detectors included on the optical temperature monitoring device 102 are disposed facing the interior of a patient's body and are directed toward the volume of the tissue to be measured for temperature. Implanting the optical temperature monitoring device 102 facing the interior of a patient's body can selectively direct emitted light toward tissues to be illuminated for temperature measurement inside the patient's body.

The optical temperature monitoring device 102 shown in FIG. 22 includes a first optical emitter 1106, a second optical emitter 1406, and a first optical detector 1108. In various embodiments, the optical temperature monitoring device 102 can be configured such that the emitted light 2214 from the first optical emitter 1106 at a first wavelength is propagated to a first depth 2210 from a surface of the optical temperature monitoring device 102, and the emitted light 2216 from the second optical emitter 1406 at a second wavelength is propagated to a second depth 2212 from a surface of the optical temperature monitoring device 102. In various embodiments, the optical temperature monitoring device 102 can be configured such that emitted light 2214 from the first optical emitter 1106 is propagated to a different depth than emitted light 2216 from the second optical emitter 1406.

In various embodiments, the depth of emitted light propagated into the tissue of the patient can be tailored by changing the distance between the first optical emitter 1106 and second optical emitter 1406, and the first optical detector 1108, where various distances suitable for use between the optical emitters and optical detectors are discussed elsewhere herein. In some embodiments, the depth of emitted light propagated into the tissue of the patient can also be tailored by selecting various wavelengths of light to be emitted by the first optical emitter 1106 and second optical emitter 1406. In some embodiments, the first wavelength and second wavelength are different wavelengths. Wavelengths suitable for use in the optical temperature monitoring devices described are discussed elsewhere herein.

Without being bound by theory, light generally follows a direct path through a medium in the absence of scattering factors. However, n more complex media where scattering is prevalent, light propagation through such media occurs by way of random scattering. Biological tissue is complex, as it is comprised of many tissue types with complex molecules and compounds distributed therein. It is believed that light propagation through biological tissue is due at least in part to scattering as caused by the difference in the index of refraction of the various molecules and compounds in the tissues, such as, but not to be limited to. By way of example, scattering of light can occur in the blood due to the difference of the index of refraction between red blood cells and plasma, and can occur in other tissues due to the difference of the index of refraction between cells, including cellular organelles, and cellular fluids, such as intracellular fluids and extracellular fluids. The resultant path for the emitted light from the optical emitter to the optical detector through biological tissues can assume a broad, arc-shaped optical path as it returns to the optical detector. The arc-shaped optical path becomes deeper as the distance between the emitter and detector increases (all other factors being equal).

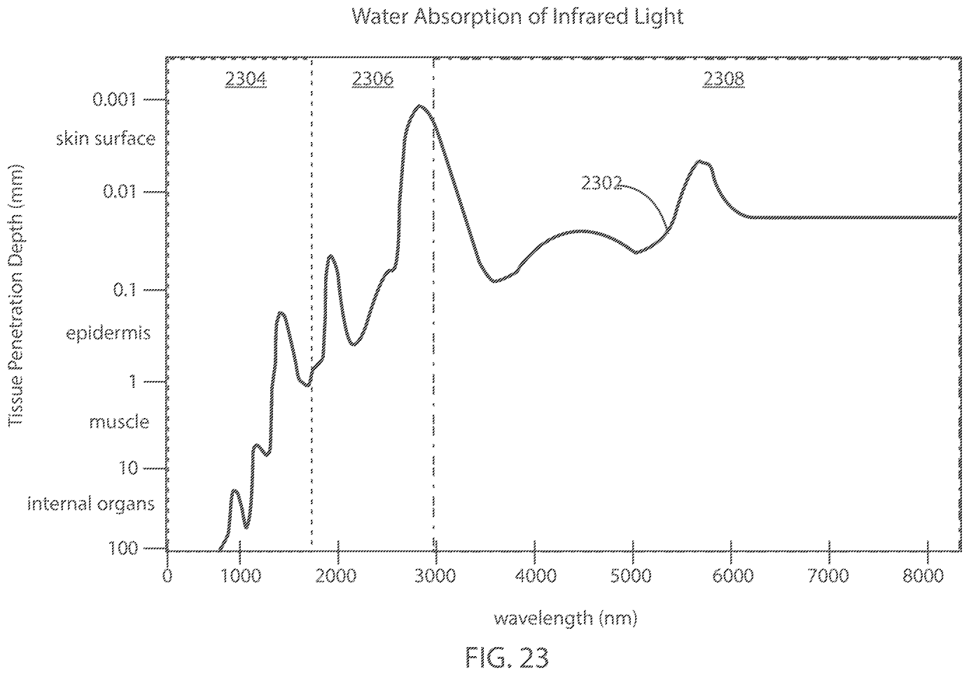

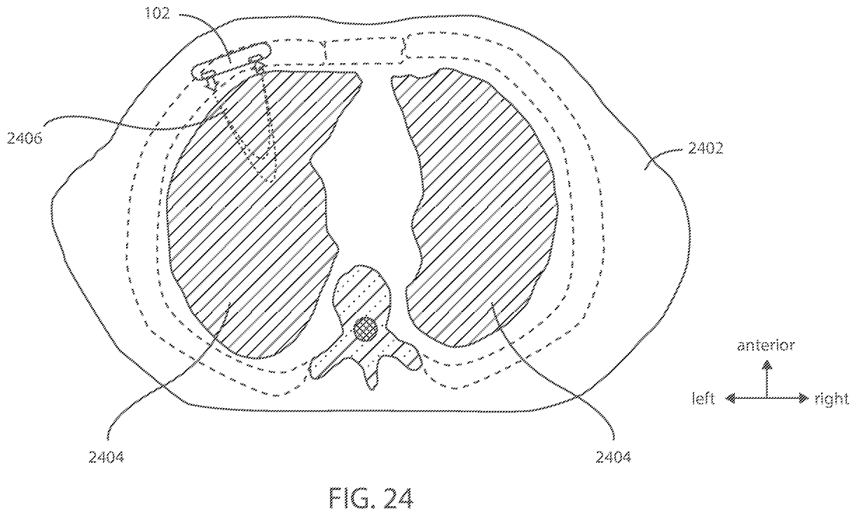

Referring now to FIG. 23, a graphical representation of water absorption of light in various tissues is shown in accordance with the embodiments herein. The tissue penetration depth in millimeters (mm) as a function of wavelength in nanometers (nm) is represented as plot 2302. At wavelengths of from 700 nm to 1500 nm, represented in section 2304 of plot 2302, the tissue penetration depth is from roughly 5 mm to 100 mm, which includes depths that penetrate within the epidermis, muscle, internal organs. At wavelengths of from 1500 nm to 3000 nm, represented in section 2306 of plot 2302, the tissue penetration depth is only from roughly 0.1 mm to 0.001 mm, which includes portions of the epidermis and the skin surface. At wavelengths greater than 3000 nm, represented in section 2308 of plot 2302, the tissue penetration depth is from 0.1 mm or less, which includes superficial portions of the epidermis and the skin surface. Thus, as can be seen, the specific wavelength chosen impacts the maximum depth of propagation through the tissue. In embodiments herein, wavelengths are selected (as also described elsewhere herein) to provide for deep tissue propagation of the emitted light.

Referring now to FIG. 24, a schematic diagram of a cross-sectional view of a human thorax 2402 is shown in accordance with the embodiments herein. The cross-sectional view of the human thorax 2402 includes the left and right lungs 2404. An optical temperature monitoring device 102 is shown positioned within an intercostal space with an optical emitter and an optical detector disposed facing a surface of the lung tissue (as merely one example of a tissue in which temperature can be measured). The optical temperature monitoring device 102 is shown disposed facing the interior of a patient's body and is directed toward the surface of the tissue in which temperature is to be measured. In some embodiments, the temperature is to be measured in tissues including, but not limited to, lung tissue, cardiovascular tissue, other chest or torso tissue, tissue of the head or neck, or portions of limbs, and the like. Implanting the optical temperature monitoring device 102 facing the interior of a patient's body can selectively direct emitted light 2406 toward tissues to be illuminated inside the patient's body.

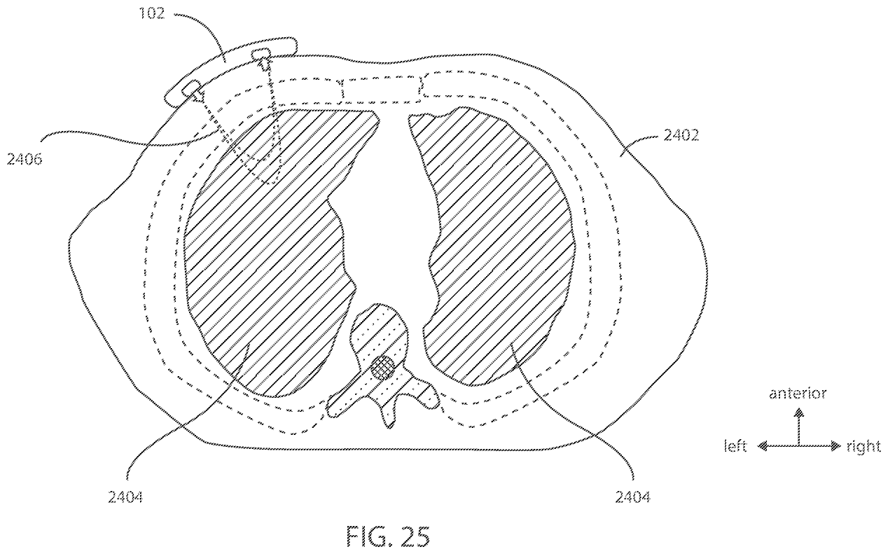

Referring now to FIG. 25, a schematic diagram of a cross-sectional view of a human thorax 2402 is shown in accordance with the embodiments herein. The cross-sectional view of the human thorax 2402 includes the left and right lungs 2404. An optical temperature monitoring device 102 having an optical emitter and optical detector is shown externally positioned on the skin and over an intercostal space. The temperature monitoring device 102 can be held in place using a wearable strap, such as described with respect to FIG. 3, or using an adhesive, or another skin mountable fixation device. The optical temperature monitoring device 102 is shown disposed facing the interior of a patient's body and is directed toward the surface of the tissue to be monitored. In some embodiments, the tissue to be measured for temperature can include lung tissue, airway tissue, cardiovascular tissue, or any other anatomical structure of interest in the body the and the like. Implanting the optical temperature monitoring device 102 with the emitter(s) and detector(s) thereof facing the interior of a patient's body can selectively direct emitted light 2406 toward tissues to be illuminated inside the patient's body.

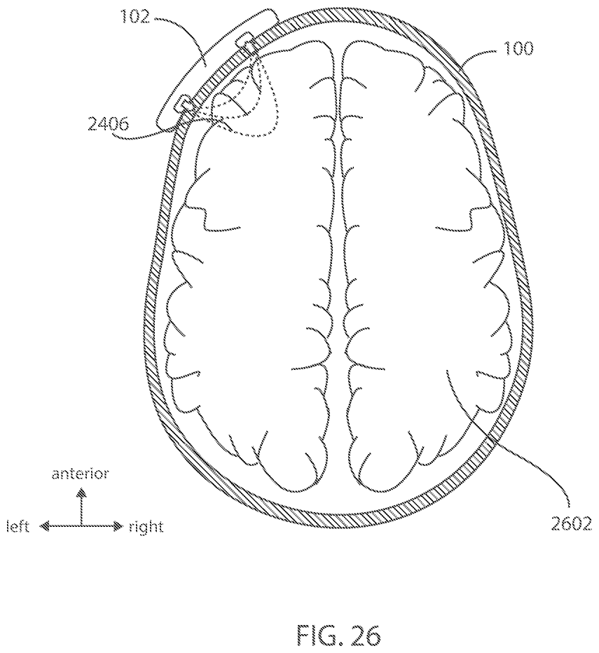

Referring now to FIG. 26, a schematic diagram of a cross-sectional view of the head of a patient 100 is shown in accordance with the embodiments herein. The cross-sectional view includes the brain 2602. An optical temperature monitoring device 102 is shown externally positioned on the head of a patient 100. The optical temperature monitoring device 102 is shown disposed facing the interior of a patient's head and is directed toward the surface of the tissue to be monitored. In some embodiments, the tissue to be monitored includes brain tissue. Placing the optical temperature monitoring device 102 with the emitter(s) and detector(s) thereof facing the interior of a patient's head can selectively direct emitted light 2406 toward tissues to be illuminated inside the patient's head. The optical temperature monitoring device 102 as shown in FIG. 26 can be a patch sensor temporarily affixed to a patient by an adhesive, or it can be a garment or strap worn on the head of a patient. As depicted in FIG. 26, the optical temperature monitoring device 102 is positioned on the anterior left side of the patient's head. However, it will be appreciated that the optical temperature monitoring device 102 can be placed anywhere on the patient's head to allow for sufficient propagation of light into the deep brain tissue of equal to or greater than 1 cm depth.

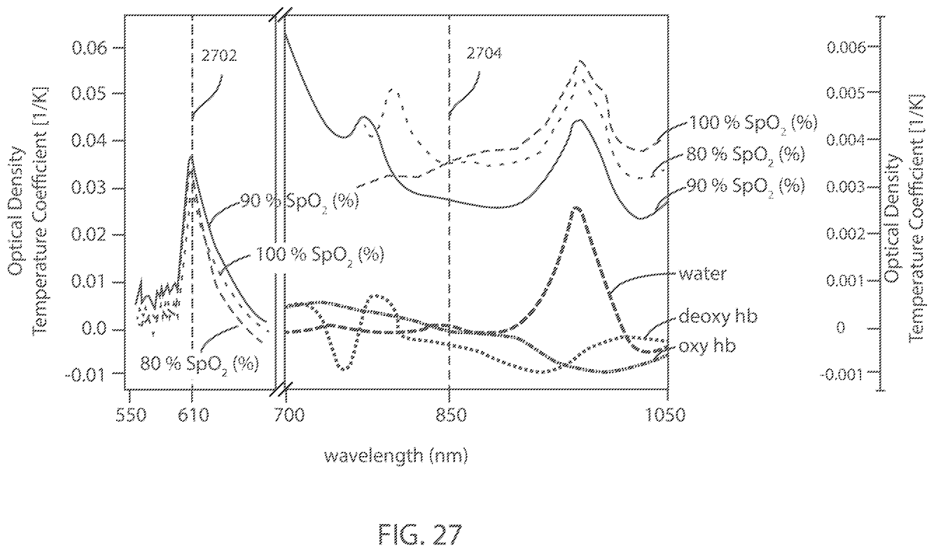

Referring now to FIG. 27, a graphical representation of the optical density temperature coefficient for blood as a function of wavelength and oxygenation status is shown in accordance with the embodiments herein. The optical density temperature coefficient for whole blood at the three different levels of oxygen saturation (100% SpO₂, 90% SpO₂, and 80% SpO₂) is plotted along with the optical density temperature coefficient for components of blood including water, oxyhemoglobin, and deoxyhemoglobin.

The optical density temperature coefficient is indicative of the relationship between the optical density and the temperature for a substance (e.g., blood). For instance, a higher optical density temperature coefficient is indicative of a stronger relationship between optical density and temperature, meaning that optical density is more sensitive to changes in temperature. Lower optical density temperature coefficient is indicative of a weaker relationship between optical density and temperature meaning that optical density is less sensitive to changes in temperature.

FIG. 27 indicates that the optical density temperature coefficient for blood is at its highest level and is reasonably immune to changes in oxygenation at wavelengths between 600 and 620 nm. The optical density temperature coefficient for blood reaches a peak at approximately 610 nm. From approximately 800 to 900 nm, the optical density temperature coefficient for blood is relatively low. The optical density temperature coefficient for water reaches a peak at approximately 960 nm accounting for a peak in the optical density temperature coefficient for blood at all oxygen saturations. Compared to the optical density temperature coefficients for blood below 700 nm, the optical density temperature coefficient for blood from 800 nm to 1100 nm is more dependent on oxygen saturation.

In various embodiments the wavelength(s) emitted by the optical emitter of the optical temperature monitoring device is selected based on the optical density temperature coefficient at that wavelength. In various embodiments, the optical emitter of the optical temperature monitoring device 102 is configured to emit light at a wavelength with a high optical density temperature coefficient. In some embodiments, the wavelength can be greater than or equal to 590, 595, 600, 605, or 610 nm. In some embodiments, the wavelength can be less than or equal to 650, 640, 630, 620, or 610 nm. In some embodiments, the wavelength can fall within a range of 590 to 650 nm, or 595 to 640 nm, or 600 to 630 nm, or 605 to 620 nm, or can be about 610 nm.

In various embodiments, the optical emitter of the optical temperature monitoring device 102 is configured to emit light at least at a first wavelength having a higher optical density temperature coefficient and at a second wavelength having a lower optical density temperature coefficient. In some embodiments, the first wavelength can fall within a range of 590 to 650 nm, or 595 to 640 nm, or 600 to 630 nm, or 605 to 620 nm, or can be about 610 nm. In some embodiments, the second wavelength can fall within a range of 800 to 850 nm, or 806 to 844 nm, or 812 to 838 nm, or 819 to 831 nm, or can be about 825 nm.

In various embodiments, the optical emitter of the optical temperature monitoring device 102 is configured to emit light at least a first wavelength having an optical density temperature coefficient with a lower dependence on blood oxygenation and a second wavelength having an optical density temperature coefficient with a higher dependence on blood oxygenation. In some embodiments, the first wavelength can fall within a range of 590 to 650 nm, or 595 to 640 nm, or 600 to 630 nm, or 605 to 620 nm, or can be about 610 nm. In some embodiments, the second wavelength can fall within a range of 800 nm to 1050 nm, or 812 nm to 1000 nm, or 825 nm to 950 nm, or 838 nm to 900 nm, or can be about 850 nm. In various embodiments, the optical emitter is configured to emit light at a first wavelength 2702 of approximately 605 nm and at a second wavelength 2704 of approximately 850 nm.

### Tissue Penetration

Spacing of the optical emitters and optical detectors along a length of the optical temperature monitoring devices herein can determine the depth of penetration of the emitted light into the surrounding tissues. By way of non-limiting example, the penetration depth of emitted light can be approximated according to the following equation:

light penetration depth=˜½(spacing between emitter and detector).

In some embodiments, the depth or propagation of the emitter light can be greater than or equal to 1 cm, 2 cm, 3 cm, 4 cm, or 5 cm, or can be an amount falling within a range between any of the foregoing. In various embodiments, the configuration of the optical temperature monitoring device can allow for the propagation of the emitted light into a patient's body and into the tissue. In some embodiments, the length between the first optical emitter and the first optical detector can be greater than or equal to 1 cm, 2 cm, 3 cm, 4 cm, 5 cm, 6 cm, 7 cm, 8 cm, 9 cm, 10 cm, 11 cm, 12 cm, 13 cm, 14 cm, or 15 cm, or can be an amount falling within a range between any of the foregoing. In various embodiments, at least 50, 60, 70, 80, 90, 95, 98, or 99 percent of the light incident upon the optical detector and used to determine the temperature of the tissue has propagated through a tissue at a depth of at least 1 cm, 2 cm, 3 cm, 4 cm, or 5 cm or more as measured from a surface of the device and/or the optical emitter(s) thereof

### Temperature Calculations

In various embodiments, at least one radiometric property of the incident light measured by the optical detector is used to determine the temperature of the tissue including, but not limited to absorption, scattering and phase. Many factors can influence the value of the measurement (e.g., tissue type, emitter energy, detector sensitivity) by the optical detector. Consequentially, a ratiometric calculation is often required to obtain an absolute temperature measurement. In various embodiments tissue temperature is dependent on a ratio of energies (E₁/E₂) detected by an optical detector of an optical temperature monitoring device.

$$Temperature ∝ \frac{{E}_{1}}{{E}_{2}}$$

The energies, E₁ and E₂ can be indicative of the optical density of the tissue. Optical absorption measured by the optical can be used to determine an optical density of the tissue. Those skilled in the art understand that a tissue with a higher optical density at a certain wavelength will absorb more of the light along the optical path from the optical emitter to the optical detector, resulting in a lower level of light incident on the optical detector at that wavelength. Using the optical density temperature coefficient as shown and described in FIG. 27 the optical density can be used to determine the temperature of the tissue. However, it will be appreciated that temperature of the tissue can be determined from a ratio of any optical parameter or parameters (e.g., absorption, scattering and phase) of the incident light on the detector. In various embodiments, the temperature is derived from a composite signal created from at least two of absorption, scattering and phase. In an exemplary embodiment, temperature is calculated using the following ratiometric equation.

$$Temperature = ( \frac{1}{{T}_{C}} * {C}_{D} * \frac{{E}_{1}}{{E}_{2}} ) + {O}_{D}$$

The ratio of energies (E1/E2) is the ratio of the energy detected by the optical detector at a first wavelength (E1) to the energy detected by the optical detector at a second wavelength (E2). T_{C} is the optical density temperature coefficient of the incident light at the first wavelength. C_{D} is the design coefficient and O_{D} is the design offset. In various embodiments, the design coefficient and the design constant can depend on the design of the optical temperature monitoring device (e.g., shape, size, and placement of optical emitter(s) and detector(s)) but are constant for all implant or placement conditions (e.g., placement and orientation in or on a patient's anatomy).

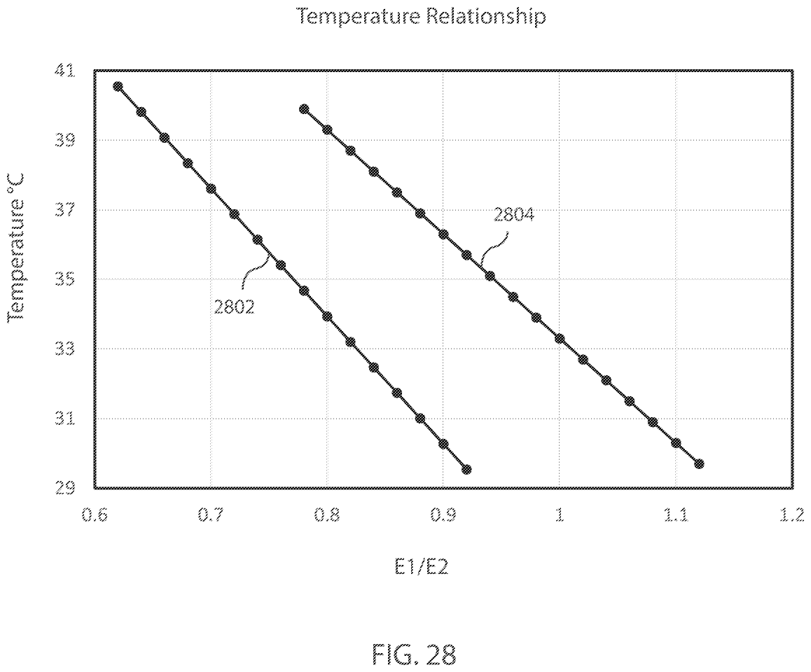

Referring now to FIG. 28, a graphical representation of temperature as a function of energy according to the above equation is shown for a first design coefficient and a second design coefficient is shown in accordance with the embodiments herein. FIG. 28 shows a first temperature vs. energy plot 2802 for a first design coefficient and a second temperature vs. energy plot 2804 for a second design coefficient. The embodiment of FIG. 28 shows two temperature vs energy plots derived from the above equation. Both plots having a first wavelength of 610 nm, a second wavelength of 850 nm, and a design offset of 63.3. The first temperature vs. energy plot 2802 corresponds to a design constant of 1.1 and the second temperature vs. energy plot 2804 corresponds to a design constant of 0.9. It is apparent from FIG. 28 and the above equation that a larger design constant results in an increased correlation between temperature and energy.

Additionally, or alternatively to making a ratiometric calculation, the temperature measurement can be calibrated with another temperature measurement. For instance, a baseline temperature measurement may be taken using an additional implantable or wearable temperature sensor and/or one or more temperature measurement techniques (e.g., rectal, ear, esophageal measurements) and the initial temperature reading of the optical temperature monitoring device can be calibrated to the baseline temperature measurement. In such an embodiment, the optical temperature monitoring device can more reliably measure temperature with a single wavelength of light.

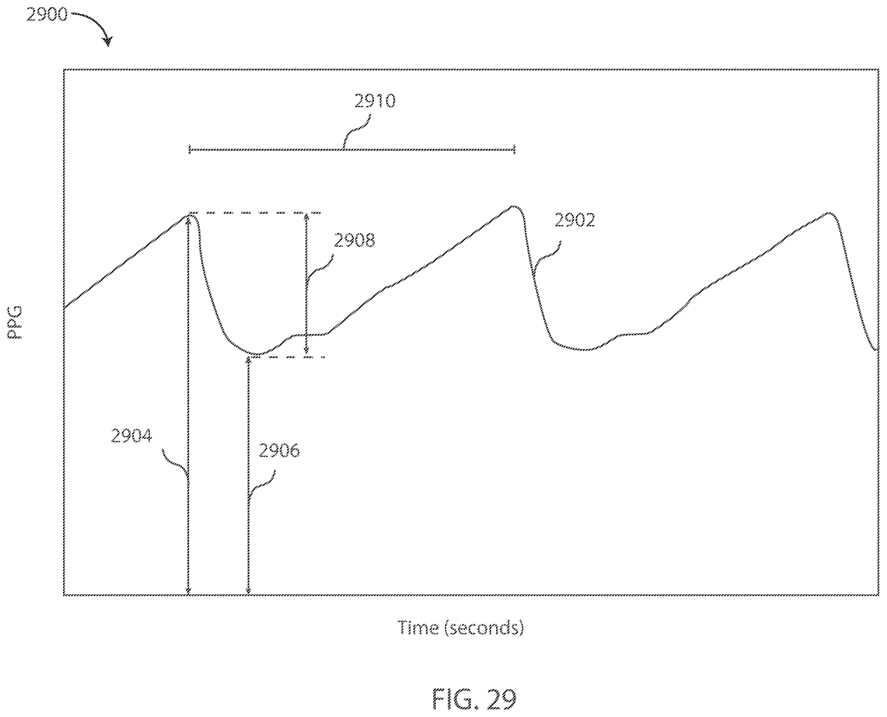

As previously discussed, the optical temperature monitoring device may further include one or more secondary sensors (e.g., pulse oximetry sensor, chemical sensor, posture sensor, and heart rate sensor). In various embodiments, the tissue temperature can be interpreted in view of at least one of the secondary signals (e.g., heart rate, respiration, circadian rhythm, and posture). In an embodiment, the tissue temperature can be measured and compared daily at the same time of day to omit variance arising from circadian rhythm. In another embodiment, the tissue temperate can be measured and compared at the same phase in the cardiac cycle to omit variance arising from the cardiac cycle (see the discussion of FIG. 29 below).

### Therapies

In some embodiments, the devices and systems herein can be configured to deliver a therapy to a patient in response to a change in temperature detected by the optical temperature monitoring devices herein. In various embodiments, the optical temperature monitoring device(s) itself can be configured to deliver a therapy to a patient. In some embodiments, implantable therapeutic devices can be implanted and used in a system in conjunction with the wearable optical temperature monitoring devices described herein. The implantable therapeutic devices can include, but not be limited to cardiac rhythm management devices, an implantable cardioverter-defibrillator (ICD), a left ventricular assist device (LVAD), a pacemaker, and the like.

### AC/DC Component Calculations

Referring now to FIG. 29, a graphical representation of an optical signal is shown in accordance with the embodiments herein. The optical signal 2900 can be indicative of the incident light measured by the optical detector of an optical temperature measurement device. As depicted by FIG. 29, the optical signal can be approximately periodic having period 2910, maximum value 2904, and minimum value 2906. In an example, the periodicity of the signal is a consequence of the systole and diastole of the cardiac cycle. During systole, the heart contracts, pushing the oxygenated blood towards the periphery of the body, resulting in an increased blood pressure. During diastole, the heart fills with blood, retracting the blood from the periphery of the body resulting in a decrease in blood pressure. Due to the absorption of light by various chromophores in blood, such as oxyhemoglobin and deoxyhemoglobin, the optical density of the tissue at certain wavelengths can increase during systole and decreases during diastole resulting, in a periodic signal with maximums and minimums. Each cycle of the signal contains a maximum value 2904 at the systolic peak and a minimum value 2906 at the diastolic valley. In another example, the periodicity of the optical signal 2900 is a consequence of inhalation and exhalation of the respiratory cycle.

The optical signal can effectively have an AC (or variable) component 2908 and a DC (or constant) component 2902. The term ‘AC’, as used herein, refers to a portion of signal that varies relatively rapidly with time. The AC component can be indicative of the portion of the signal originating by pulsations in a patient's blood during each heartbeat. The term ‘DC’, as used herein, refers to portions of the signal that are relatively invariant with time. Alternatively, the term ‘DC’, as used herein, refers to the mean value of the signal.

In various embodiments, both an AC component and a DC component of the light detected by the optical detector can be used to determine a property of the tissue (e.g., temperature). In various embodiments, the property can be derived from a ratio of the AC and DC components of the signal as measured by the optical detector. In various embodiments, the property can be derived from a ratio of the AC and DC components of the signal measured by the optical detector at two distinct wavelengths. For instance, the property can be derived using a ratio of ratios (RoR) calculation according to the following calculation:

$$Temperature ∝ \frac{A {C}_{1} / {DC}_{1}}{A {C}_{2} / {DC}_{2}}$$

where AC₁ is the AC component of the signal measured by the optical detector at the first wavelength; DC₁ is the DC component of the signal measured by the optical detector at the first wavelength; AC₂ is the AC component of the signal measured by the optical detector at the second wavelength; and DC₂ is the DC component of the signal measured by the optical detector at the second wavelength.

### Emitter/Detector Characteristics

In some embodiments, the one or more optical emitters can include solid state light sources such as GaAs, GaAlAs, GaAlAsP, GaAlP, GaAsP, GaP, GaN, InGaAlP, InGaN, ZnSe, or SiC light emitting diodes or laser diodes that excite the sensing one or more optical detectors at or near the wavelength of maximum absorption for a time sufficient to emit a return signal. However, it will be understood that in some embodiments the wavelength of maximum absorption/reflection varies as a function of the optical path from the one or more optical emitters to the one or more optical detectors.

In some embodiments, the one or more optical emitters can include other light emitting components including incandescent components. In some embodiments, the optical emitters can include a waveguide. The optical emitters can also include one or more filters such as bandpass filters, high pass filter, low pass filter, and/or other components such as antireflection elements, and/or focusing optics.

In some embodiments, the one or more optical emitters can include a plurality of LEDs with bandpass filters, each of the LED-filter combinations emitting at a different center frequency. According to various embodiments, the LEDs can operate at different center-frequencies, sequentially turning on and off during a measurement. As multiple different center-frequency measurements are made sequentially, a single unfiltered optical detector can be used in some embodiments. However, in some embodiments, a polychromatic source can be used with multiple detectors that are each bandpass filtered to a particular center frequency.

The one or more optical detectors can be configured to receive light from the optical emitters. In an embodiment, the optical detectors can include a component to receive light. By way of example, in some embodiments, the optical detectors can include a charge-coupled device (CCD). In other embodiments, the optical detectors can include a photodiode, a junction field effect transistor (JFET) type optical sensor, or a complementary metal-oxide semiconductor (CMOS) type optical sensor. In some embodiments, the optical detectors can include an array of optical detecting components. In some embodiments, the optical detectors can include a waveguide. The one or more optical detectors can also include one or more bandpass filters and/or other components such as antireflection elements, optical barriers, and/or focusing optics. In some embodiments, the optical detectors can include one or more photodiode detectors, each with an optical bandpass filter tuned to a specific wavelength range.

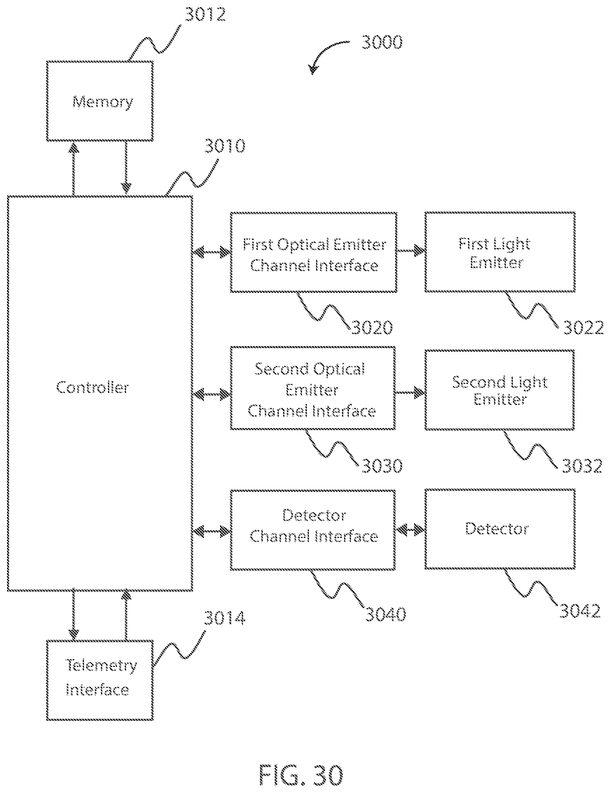

Referring now to FIG. 30, a schematic block diagram of some components 3000 of an optical temperature monitoring device is shown in accordance with various embodiments herein. It will be appreciated that some embodiments can include additional elements beyond those shown in FIG. 30. In addition, some embodiments may lack some elements shown in FIG. 30. The implantable sensing devices herein can gather information through one or more detecting channels. A controller 3010 can communicate with a memory 3012 via a bidirectional data bus. It will be appreciated that controller 3010 can include one or more microprocessors. The memory 3012 can include read-only memory (ROM) or random-access memory (RAM) for program storage and RAM for data storage, or any combination thereof. The implantable medical device can include one or more optical emitters 3022, 3032, one or more detectors 3042, or one or more other sensors (not pictured). The one or more other sensors, but are not to be limited to, a pulse oximetry sensor, a chemical sensor, a posture sensor, or a heart rate sensor.

Each optical emitter 3022, 3032 is communicatively coupled to an optical emitter channel interface 3020, 3030. Each detector 3042 is communicatively coupled to a detector channel interface 3040. Each other sensor is communicatively coupled to a separate and other sensor channel interface (not pictured). Each of the optical emitter channel interfaces 3020, 3030, the detector channel interface 3040, and any of the other sensor channel interfaces can communicate with controller 3010.

The first optical emitter channel interface 3020, the second optical emitter channel interface 3030, and the detector channel interface 3040 can each include various components such as analog-to-digital converters for digitizing signal inputs, sensing amplifiers, registers which can be written to by the control circuitry to adjust the gain and threshold values for the sensing amplifiers, source drivers, modulators, demodulators, multiplexers, and the like. A telemetry interface 3014 is also provided for communicating with external devices such as a programmer, a home-based unit, and/or a mobile unit (e.g., a cellular phone, portable computer, etc.), implanted devices such as a pacemaker, cardioverter-defibrillator, loop recorder, and the like.

### Methods

Many different methods are contemplated herein, including, but not limited to, methods of making, methods of using, methods of measuring temperature, and the like. Aspects of system/device operation described elsewhere herein can be performed as operations of one or more methods in accordance with various embodiments herein.

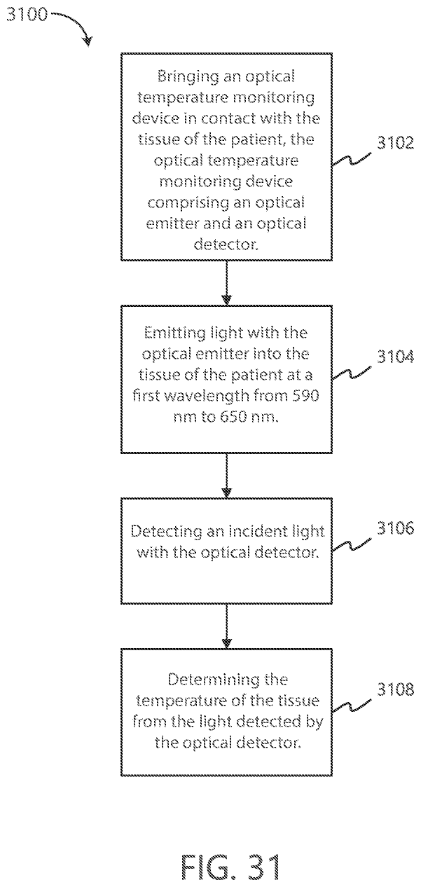

Referring now to FIG. 31, a method 3100 of measuring a temperature of the tissue in a patient is shown in accordance with the embodiments herein. The method can include bringing an optical temperature monitoring device in contact with the tissue of the patient in operation 3102, where the optical temperature monitoring device can include an optical emitter and an optical detector. The method can include emitting light with the optical emitter into the tissue of the patient at a first wavelength in operation 3104, where the first wavelength is from 590 nm to 650 nm and the light from the optical emitter propagates at a depth of at least 1 cm into the tissue as measured from a surface of the optical temperature monitoring device. The method can include detecting incident light with the optical detector in operation 3106. The method can include determining the temperature of the tissue from the light detected by the optical detector in operation 3108.

In an embodiment, the method can further include implanting the optical temperature monitoring device subcutaneously in the patient's tissue.

In another embodiment, the method can further include implanting the optical temperature monitoring device within or proximal to a muscle, a vessel, or an organ of a patient.

In an embodiment of the method, implanting the optical temperature monitoring device subcutaneously in the patient's tissue comprises orienting the optical temperature monitoring device such that optical emitter and the optical detector face internally relative to the skin of the patient.

In an embodiment, the method can further include emitting light into the tissue of the patient using a second optical emitter at a second wavelength.

In an embodiment of the method, the optical emitter is disposed from 1 cm to 10 cm away from the optical detector.

In an embodiment, the method can further include measuring at least one of heart rate, respiration rate, circadian rhythm, menstrual cycle, and posture.

In an embodiment, the method can further include delivering a therapy to the patient.

In an embodiment, the method can further include measuring at least one of absorption, light scattering, and phase with the optical detector.

### Systems

The devices herein can be used in various systems including multiple optical temperature monitoring devices and secondary sensors, including, but not limited to, methods of making, methods of using, and the like. Aspects of optical temperature monitoring devices described elsewhere herein can be included of one or more embodiments of a system in accordance with various embodiments herein.

In an embodiment, a sensor system is included for detecting tissue temperature of a patient. The optical temperature monitoring system can include a housing. The optical temperature monitoring system can include a first optical temperature monitoring device, the first optical temperature monitoring device including an optical emission assembly, wherein the optical emission assembly includes a first optical emitter configured to emit light at a first wavelength from 590 nm to 650 nm and a second optical emitter configured to emit light at a second wavelength. The system can further include an optical detector, where the optical detector is configured to detect incident light. In the systems described herein, the light from the first optical emitter and the second optical emitter can be configured to propagate through tissue. In the systems described herein, the detected incident light detected by the optical detector can be used to determine temperature of the tissue. In the systems described herein, the optical temperature monitoring system is configured to be implanted in a subcutaneous tissue of a patient.

In an embodiment of the system, at least one of absorption, scattering and phase of the light detected by the optical detector is used to determine the temperature of the tissue.

In an embodiment of the system the absorption is used to determine an optical density of the tissue and the optical density is used to determine a temperature of the tissue.

In an embodiment of the system the temperature of the tissue is a function of the ratio of the detected light originating from the first optical emitter and the detected light originating from the second optical emitter.

In an embodiment of the system both an AC component and an DC component of the light detected by the optical detector are used to determine the temperature of the tissue.

In an embodiment of the system, the optical emitter and the optical detector are disposed on a substantially planar surface.

In an embodiment of the system, the optical emitter is disposed 1 cm to 10 cm away from the optical detector.

In an embodiment of the system, the optical temperature monitoring device of claim 1, wherein the light from the optical emitter propagates a depth of 1 cm to 5 cm into the tissue as measured from a surface of the optical temperature monitoring device.

In an embodiment of the system, the system includes at least one of a pulse oximetry sensor, chemical sensor, posture sensor, and heart rate sensor.

In an embodiment of the system, the temperature of the tissue is interpreted in view of at least one of heart rate, respiration, circadian rhythm, and posture.

In an embodiment of the system, at least one of the optical emitter and the optical detector are disposed on a flexible member extending from the housing.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application were specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An optical temperature monitoring device comprising:
an optical emitter, wherein the optical emitter is configured to emit light at a first wavelength;
wherein the first wavelength is from 100 nm to 2000 nm;
an optical detector, wherein the optical detector is configured to detect incident light;
wherein the optical temperature monitoring device is configured so that the light from the optical emitter propagates at a depth of at least 1 cm through tissue as measured from a surface of the optical temperature monitoring device and back to the optical detector; and
wherein the incident light detected by the optical detector is used to determine a temperature of the tissue.

2. The optical temperature monitoring device of claim 1, wherein the first wavelength is from 590 nm to 650 nm.

3. The optical temperature monitoring device of claim 1, wherein the optical temperature monitoring device is configured to be implanted in a patient.

4. The optical temperature monitoring device of claim 1, wherein at least one of absorption, scattering and phase of the incident light detected by the optical detector is used to determine the temperature of the tissue.

5. The optical temperature monitoring device of claim 4, wherein the absorption is used to determine an optical density of the tissue and the optical density is used to determine the temperature of the tissue.

6. The optical temperature monitoring device of claim 1, wherein both an AC component and an DC component of the light detected by the optical detector are used to determine the temperature of the tissue.

7. The optical temperature monitoring device of claim 1, further comprising a second optical emitter, wherein the second optical emitter is configured to emit the light at a second wavelength.

8. The optical temperature monitoring device of claim 7, wherein the second wavelength is from 750 nm to 1500 nm.

9. The optical temperature monitoring device of claim 7, wherein the second wavelength has an optical density temperature coefficient that is less than the first wavelength.

10. The optical temperature monitoring device of claim 7, wherein the temperature of the tissue is determined from a ratio of a parameter of the incident light on the detector originating from the light emitted at the first wavelength and a parameter of the incident light on the detector originating from the light emitted at the second wavelength.

11. The optical temperature monitoring device of claim 1, further comprising:
a housing; and
a flexible member;
wherein at least one of the optical emitter and the optical detector are disposed on the flexible member extending from the housing; and
wherein the flexible member extends outward from the housing.

12. The optical temperature monitoring device of claim 1, wherein the optical emitter and the optical detector are disposed on a substantially planar surface.

13. The optical temperature monitoring device of claim 1, wherein the optical emitter is disposed from 1 cm to 10 cm away from the optical detector.

14. The optical temperature monitoring device of claim 1, further comprising at least one of a pulse oximetry sensor, chemical sensor, posture sensor, and heart rate sensor.

15. The optical temperature monitoring device of claim 14, wherein the temperature of the tissue is interpreted in view of at least one of heart rate, respiration, circadian rhythm, and posture.

16. The optical temperature monitoring device of claim 1,
the optical emitter comprising a first optical emitter element and a second optical emitter element; and
wherein the first optical emitter element is spaced a distance away from the second optical emitter element.

17. A method of measuring a temperature of the tissue in a patient comprising:
bringing an optical temperature monitoring device in contact with the tissue of the patient, the optical temperature monitoring device comprising an optical emitter and an optical detector; and
emitting light with the optical emitter into the tissue of the patient at a first wavelength;
wherein the first wavelength is from 590 nm to 650 nm;
wherein the light from the optical emitter propagates at a depth of 1 cm to 5 cm into the tissue as measured from a surface of the optical temperature monitoring device;
detecting incident light with the optical detector; and
determining the temperature of the tissue from the light detected by the optical detector.

18. The method of claim 17, further comprising implanting the optical temperature monitoring device in the patient's tissue and orienting the optical temperature monitoring device such that optical emitter and the optical detector face internally relative to the skin of the patient.

19. The method of claim 17, further comprising emitting light into the tissue of the patient using a second optical emitter at a second wavelength.

20. The method of claim 17, wherein the optical emitter is disposed from 1 cm to 10 cm away from the optical detector.
